(19) United States
(12) Patent Application Publication
(10) Pub. No.: US 2004/0025202 A1
(43) Pub. Date: Feb. 5, 2004

(54) Nucleic acid molecules associated with oil in plants

(76) Inventors: Cathy C. Laurie, Winchester, MA (US); Monica Ravanello, Vacaville, CA (US); Thomas Savage, Sacramento, CA (US); John R. Ledeaux, Creve Coeur, MO (US); James A. Rogers, Hingham, MA (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/389,566

(22) Filed: Mar. 14, 2003

Related U.S. Application Data
(60) Provisional application No. 60/365,301, filed on Mar. 15, 2002.
(60) Provisional application No. 60/391,786, filed on Jun. 26, 2002.
(60) Provisional application No. 60/392,018, filed on Jun. 26, 2002.

(51) Int. Cl. A01H 1/00; C12N 15/82; A01H 5/00

(52) U.S. Cl. 800/281; 800/320.1

(57) ABSTRACT

Polynucleotides that encode proteins associated with oil content in plants are useful in constructs to make transgenic plants, e.g., maize or soybean, with desirable oil content phenotype and progeny of any generation derived from the fertile transgenic plants. Markers associated with oil content QTL are useful in breeding for plants with desired oil content.

### REFERENCE TO RELATED APPLICATIONS

This application claims priority to U.S. Provisional Applications 60/365,301 filed Mar. 15, 2002, No. 60/391,786 filed Jun. 25, 2002 and No. 60/392,018 filed Jun. 26, 2002, herein incorporated by reference in their entirety.

### INCORPORATION OF SEQUENCE LISTING

Two copies of the sequence listing (Seq. Listing Copy 1 and Seq. Listing Copy 2) and a computer-readable form of the sequence listing, all on CD-ROMs, each containing the file named 52900.ST25.txt, which is 9,302,045 bytes (measured in MS-DOS) and was created on Mar. 13, 2003, are herein incorporated by reference.

### INCORPORATION OF TABLES

Two copies of Table 1 (Table 1 Copy 1 and Table 1 Copy 2) all on CD-ROMs, each containing the file named 52900_Table 1.txt, which is 226,395 bytes (measured in MS-DOS) and was created on Mar. 14, 2003, are herein incorporated by reference.

### FIELD OF THE INVENTION

Disclosed herein are inventions in the field of plant molecular biology, plant genetics and plant breeding. More specifically disclosed are nucleic acid and amino acid molecules associated with oil in plants, particularly oil in maize. Also disclosed are genetic markers for such nucleic acid molecules and genes and QTLs associated with oil in maize. Such markers are useful for discovery and isolation of genes useful in enhancing the level of oil in plants and for molecular breeding of maize with enhanced levels of oil. Also disclosed are transgenic plants with altered expression of one or more genes associated with oil.

### BACKGROUND OF THE INVENTION

Maize, Zea mays L., is one of the major crops grown worldwide as a primary source for animal feed, human food and industrial purposes. Maize plants with improved agronomic traits, such as yield or pest resistance; improved quality traits such as oil, protein or starch quality or quantity; or improved processing characteristics, such as extractability of desirable compounds, are desirable for both the farmer and consumer of maize and maize derived products. The ability to breed or develop transgenic plants with improved traits depends in part on identification of genes associated with a trait. The unique maize sequences disclosed herein may be useful as mapping tools to assist in plant breeding and in designing transgenic plants. Homologous sequences in plant species other than maize and in fungi, algae and bacteria may be useful to confer novel phenotypes in transgenic maize and other oil-producing plants.

Increases in the oil content of maize seeds can be achieved by altering the expression of one or more genes that encode a protein that functionally increases oil production or storage. Effective changes in expression may include constitutive increases, constitutive decreases or alterations in the tissue-specific pattern of expression. See, for instance, U.S. Pat. No. 6,268,550, which discloses that a higher oil content soybean is associated with a twofold increase in acetyl CoA carboxylase (ACCase) activity during early to mid stages of development when compared with a low oil content soybean. In view of a correlation of increased expression of the ACCase gene with an increase in the oil content of the seed, it is predicted that over expression of the ACCase enzyme is likely to lead to an increase in the oil content of the plants and seeds. Because metabolic pathways affecting oil production and storage are complex and controlled by a large number of enzymes and transcription factors, there is a need to discover and modulate the expression of other genes associated with oil.

Polymorphisms are useful as genetic markers for genotyping applications in the agriculture field, e.g., in plant genetic studies and commercial breeding. See for instance U.S. Pat. Nos. 5,385,835; 5,492,547 and 5,981,832, the disclosures of all of which are incorporated herein by reference. The highly conserved nature of DNA combined with the rare occurrences of stable polymorphisms provide genetic markers that are both predictable and discerning of different genotypes. Among the classes of existing genetic markers are a variety of polymorphisms indicating genetic variation including restriction-fragment-length polymorphisms (RFLPs), amplified fragment-length polymorphisms (AFLPs), simple sequence repeats (SSRs), single nucleotide polymorphisms (SNPs), and insertion/deletion polymorphisms (Indels). Because the number of genetic markers for a plant species is limited, the discovery of additional genetic markers associated with a trait will facilitate genotyping applications including marker-trait association studies, gene mapping, gene discovery, marker-assisted selection, and marker-assisted breeding. Evolving technologies make certain genetic markers more amenable for rapid, large scale use. For instance, technologies for SNP detection indicate that SNPs may be preferred genetic markers.

### SUMMARY OF THE INVENTION

This invention provides genes that have been identified as being associated with high oil in maize. An aspect of this invention provides homologs of such genes from a variety of other plant species and other organisms, e.g., fungi, algae and bacteria. Nucleic acid molecules derived from such genes and homologous genes that encode proteins that are effective in the production or storage of oil in plant seeds are useful in other aspects of this invention, e.g., DNA constructs for producing transgenic plants and seed with higher or lower oil. Thus, a particular aspect of this invention is transgenic plant seed having in its genome a recombinant DNA construct comprising at least one oil-associated gene of this invention operably linked to a promoter that is functional in the plant to transcribe the oil-associated gene. In one preferred aspect of this invention such transgenic plant seeds can grow into plants having enhanced seed oil as compared to wild type. Conversely, an alternative aspect of this invention employs gene suppression technology, e.g., RNAi gene suppression, to provide transgenic plant seeds having a recombinant DNA construct which includes DNA effective for suppression of an oil-associated gene. Such seeds can be grown into plants having reduced seed oil as compared to wild type. Alternatively, the suppression of the oil-associated gene could lead to plants with increased seed oil compared to wild type, depending on the action of the gene. In another aspect of this invention, the oil-associated gene can be over expressed or can be expressed in a tissue- or stage-specific manner to alter the oil content or properties of the plant seed as compared to wild type.

Another aspect of this invention provides hybrid maize seed that is produced by crossing two parental maize lines where at least one of the parental maize lines is a transgenic maize line that has in its genome a recombinant DNA construct for producing transgenic maize with enhanced seed oil as compared to its parents, e.g., its non-transgenic ancestors. Such hybrid maize seed will have a recombinant DNA construct comprising at least one oil-associated gene of this invention operably linked to a promoter that is functional in maize to transcribe the oil-associated gene. Still another aspect of this invention provides hybrid maize seed that can produce maize plants characterized by agronomic traits of seed oil level, yield and standability. Preferably, seed oil level is greater than seed oil level in said closest non-transgenic parental lines and, even more preferably, there is essentially no reduction in yield and standability traits in said maize plants as compared to yield and standability traits for said closest non-transgenic parental lines.

Still another aspect of this invention provides methods of producing hybrid maize plants having enhanced levels of seed oil production or seed oil storage as compared to the closest non-transgenic ancestor maize lines. Such methods comprise producing a transgenic maize plant having in its genome a recombinant DNA construct comprising at least one oil-associated gene of this invention operably linked to a promoter that is functional in maize to transcribe the oil-associated gene. Such methods further comprise crossing transgenic progeny of transgenic maize plants with at least one other maize plant to produce hybrid maize plants having enhanced levels of seed oil production.

Yet another aspect of this invention relates to a method for producing vegetable oil by growing and harvesting oil from plants of this invention.

This invention also provides maize oil markers that have been identified as statistically significant in associating with high oil in maize. Such markers are especially useful in methods of this invention relating to breeding maize for high oil. More particularly, this invention provides a method of breeding maize comprising selecting from a breeding population of maize plants a selected maize plant with higher oil than other maize plants in the breeding population based on allelic polymorphisms associated by linkage disequilibrium to a higher seed oil-related trait, where the selected maize plant has 1 or more higher oil alleles linked to a maize oil marker of this invention. The maize oil markers are also useful in a method of breeding maize comprising selecting a maize line having a haplotype characterized by the maize oil markers. The maize oil markers are also useful in methods of this invention for identifying other polymorphic maize DNA loci, which are useful for genotyping between at least two varieties of maize. More particularly such a method comprises identifying a locus comprising at least 20 consecutive nucleotides that are linked to a maize oil marker locus of this invention. Thus, a further aspect of this invention provides methods of breeding maize comprising selecting a maize line having a polymorphism associated by linkage disequilibrium to a seed oil-related trait locus where such polymorphism is linked to a maize oil marker of this invention.

Aspects of this invention related to maize oil markers are isolated nucleic acid molecules that are useful for detecting a polymorphism associated with oil in maize, e.g., molecules that are known in the art as PCR primers and hybridization probes for using the markers in genotyping.

### DETAILED DESCRIPTION OF PREFERRED EMBODIMENTS

As used herein certain terms are defined as follows.

An “oil-associated gene” means a nucleic acid molecule comprising at least a functional part of the open reading frame of a gene (or a homolog thereof) that either overlaps with, or is associated by linkage disequilibrium with, any one or more of the 186 genomic amplicons of SEQ ID NO:1 through SEQ ID NO:186, which contain markers having a statistically significant association with an oil trait. More particularly, oil-associated genes are found in the group consisting of:

(a) on maize chromosome 1 the genes characterized by nucleic acid sequences of SEQ ID NO: 266, 291, 340, 255, 265, 187, 322, 243, 261, 244, 200, 248, 228, 251, 319, 321, 290, 249, 263, 299, 196, 242, 279, 306, 338, and 233; a gene having DNA that overlaps, or is associated by linkage disequilibrium with, the marker amplicon defined by SEQ ID NO: 185; genes encoding proteins having an amino acid sequence selected from the group consisting of SEQ ID NO: 425, 450, 499, 414, 424, 346, 481, 402, 420, 403, 359, 407, 387, 410, 478, 480, 449, 408, 422, 458, 355, 401, 438, 465, 497, and 392; and homologs thereof selected from plants, fungi, algae and bacteria;

(b) on maize chromosome 2 the genes characterized by nucleic acid sequences of SEQ ID NO: 234, 259, 296, 285, 283, 208, 205, 282, 305, 190, 288, 339, 317, 303, 189, 220, 293, 267, 188, and 281; a gene having DNA that overlaps, or is associated by linkage disequilibrium with, the marker amplicon defined by SEQ ID NO: 180; genes encoding proteins having an amino acid sequence selected from the group consisting of SEQ ID NO:393, 418, 455, 444, 442, 367, 364, 441, 464, 349, 447, 498, 476, 462, 348, 379, 452, 426, 347, and 440; and homologs thereof selected from plants, fungi, algae and bacteria;

(c) on maize chromosome 3 the genes characterized by nucleic acid sequences of SEQ ID NO: 272, 204, 239, 270, 307, 217, 312, 310, 229, 194, 219, 225, 334, 212, 240, 202, 315, and 326; genes having DNA that overlaps, or is associated by linkage disequilibrium with, a marker amplicon defined by SEQ ID NO: 165, 169, 172, 164, 167, and 166; genes encoding proteins having an amino acid sequence selected from the group consisting of SEQ ID NO: 431, 363, 398, 429, 466, 376, 471, 469, 388, 353, 378, 384, 493, 371, 399, 361, 474, and 485; and homologs thereof selected from plants, fungi, algae and bacteria;

(d) on maize chromosome 4 the genes characterized by nucleic acid sequences of SEQ ID NO: 222, 345, 206, 195, 252, 300, 287, 298, 295, 273, 337, 238, 214, and 333; genes having DNA that overlaps, or is associated by linkage disequilibrium with, a marker amplicon defined by SEQ ID NO: 182, 184, and 176; genes encoding proteins having an amino acid sequence selected from the group consisting of SEQ ID NO: 381, 504, 365, 354, 411, 459, 446, 457, 454, 432, 496, 397, 373, and 492; and homologs thereof selected from plants, fungi, algae and bacteria;

(e) on maize chromosome 5 the genes characterized by nucleic acid sequences of SEQ ID NO: 221, 309, 211, 308, 213, 271, 241, 332, 323, 227, 250, 275, and 235; genes having DNA that overlaps, or is associated by linkage disequilibrium with, a marker amplicon defined by SEQ ID NO: 168, 174, 186, and 181; genes encoding proteins having an amino acid sequence selected from the group consisting of SEQ ID NO: 380, 468, 370, 467, 372, 430, 400, 491, 482, 386, 409, 434, and 394; and homologs thereof selected from plants, fungi, algae and bacteria;

(f) on maize chromosome 6 the genes characterized by nucleic acid sequences of SEQ ID NO: 343, 280, 247, 231, 193, 277, 237, 274, 304, 276, 331, 191, 294, 335, 344, 218, 198, 210, 316, 236, 254, 253; genes encoding proteins having an amino acid sequence selected from the group consisting of SEQ ID NO: 502, 439, 406, 390, 352, 436, 396, 433, 463, 435, 490, 350, 453, 494, 503, 377, 357, 369, 475, 395, 413, and 412; and homologs thereof selected from plants, fungi, algae and bacteria;

(g) on maize chromosome 7 the genes characterized by nucleic acid sequences of SEQ ID NO: 264, 232, 257, 278, 197, 268, 245, 256, 192, 284, 329, 209, 260, and 230; genes having DNA that overlaps, or is associated by linkage disequilibrium with, a marker amplicon defined by SEQ ID NO: 177, 163, 162, and 175; genes encoding proteins having an amino acid sequence selected from the group consisting of SEQ ID NO: 423, 391, 416, 437, 356, 427, 404, 415, 351, 443, 488, 368, 419, and 389; and homologs thereof selected from plants, fungi, algae and bacteria;

(h) on maize chromosome 8 the genes characterized by nucleic acid sequences of SEQ ID NO: 262, 302, 318, 223, 292, 224, 328, 313, 289, 269, 286, 314, 203, 301, 207 and 327; genes having DNA that overlaps, or is associated by linkage disequilibrium with, a marker amplicon defined by SEQ ID NO: 171, 179, and 170; genes encoding proteins having an amino acid sequence selected from the group consisting of SEQ ID NO: 421, 461, 477, 382, 451, 383, 487, 472, 448, 428, 445, 473, 362, 460, 366, and 486; and homologs thereof selected from plants, fungi, algae and bacteria;

(i) on maize chromosome 9 the genes characterized by nucleic acid sequences of SEQ ID NO: 226, 320, 336, 215, 341, 199, 201, and 246; genes having DNA that overlaps, or is associated by linkage disequilibrium with, a marker amplicon defined by SEQ ID NO: 178, 183, and 173; genes encoding proteins having an amino acid sequence selected from the group consisting of SEQ ID NO: 385, 479, 495, 374, 500, 358, 360, and 405; and homologs thereof selected from plants, fungi, algae and bacteria;

(j) on maize chromosome 10 the genes characterized by nucleic acid sequences of SEQ ID NO: 325, 258, 297, 324, 330, and 311; genes encoding proteins having an amino acid sequence selected from the group consisting of SEQ ID NO: 484, 417, 456, 483, 489, and 470; and homologs thereof selected from plants, fungi, algae and bacteria;

(k) genes characterized by the unmapped nucleic acid sequences of SEQ ID NO: 216 and 342; genes encoding proteins having an amino acid sequence selected from the group consisting of SEQ ID NO: 375 and 501; and homologs thereof selected from plants, fungi, algae and bacteria;

(l) homologs of maize oil-associated genes that encode a protein identified in Table 1, which have the amino acid sequences of SEQ ID NO: 505 through SEQ ID NO: 2459;

(m) nucleic acid molecules comprising oligonucleotides of at least 40 consecutive nucleic acid residues of a gene in sections (a) through 0) and having at least 60%, more preferably at least 70%, even more preferably at least 80%, and most preferably at least 90% identity with a same length fragment of said gene; and

(n) nucleic acid molecules encoding polypeptides having an amino acid sequence that has at least 80% similarity to an amino acid sequence of a protein in sections (a) through (l).

An “allele” means an alternative sequence at a particular locus; the length of an allele can be as small as 1 nucleotide base but is typically larger. Allelic sequence can be amino acid sequence or nucleic acid sequence.

A “locus” is a short sequence that is usually unique and usually found at one particular location by a point of reference, e.g., a short DNA sequence that is a gene, or part of a gene or intergenic region. A locus of this invention can be a unique PCR product. The loci of this invention are polymorphic between certain individuals.

“Genotype” means the specification of an allelic composition at one or more loci within an individual organism. In the case of diploid organisms, there are two alleles at each locus; a diploid genotype is said to be homozygous when the alleles are the same, and heterozygous when the alleles are different.

“Consensus sequence” means

(a) a constructed DNA sequence that identifies SNP and Indel polymorphisms in alleles at a locus. Consensus sequence of a polymorphic locus can be based on either strand of DNA at the locus and states the nucleotide base of either one of each SNP in the locus and the nucleotide bases of all Indels in the locus. Thus, although a consensus sequence of a polymorphic locus may not be a copy of an actual DNA sequence, a consensus sequence is useful for precisely designing primers and probes for actual polymorphisms in the locus.

(b) a conserved amino acid sequence of part or all of the proteins encoded by homogolous genes.

“Homolog” of an oil-associated gene as used herein means a gene from the same or a different organism that performs the same biological function as the oil-associated gene. An orthologous relation between two organisms is not necessarily manifest as a one-to-one correspondence between two genes, because a gene can be duplicated or deleted after organism phylogenetic separation, such as speciation. So for a given gene, there may be no ortholog or more than one ortholog. Other complicating factors include alternatively spliced transcripts from the same gene, limited gene identification, redundant copies of the same gene with different sequence lengths or corrected sequence. A local sequence alignment program, e.g., BLAST, can be used to search a database of sequences to find similar sequences, and the summary Expectation value (E-value) can be used to measure the sequence base similarity. Because query results with the best E-value for a particular organism may not necessarily be an ortholog or the only ortholog, it is necessary to use a reciprocal BLAST search to filter the hit sequences with significant E-values before calling them orthologs. The reciprocal BLAST entails search of the significant hits against a database of genes from the base organism that are similar to the query gene. A hit is a likely ortholog when the reciprocal BLAST's best hit is the query gene itself or is one of the duplicated genes of the query gene after speciation. Some skilled in the art may argue that what is called a homolog is in fact an ortholog or a paralog. Regardless, the term homolog is used herein to describe genes that are assumed to have functional similarity by inference from sequence base similarity. A detailed procedure is set forth below in Example 3.

“Phenotype” means the detectable characteristics of a cell or organism that are a manifestation of gene expression.

“Marker” means a polymorphic sequence. A “polymorphism” is a variation among individuals in sequence, particularly in DNA sequence. Useful polymorphisms include a single nucleotide polymorphisms (SNPs) and insertions or deletions in DNA sequence (Indels).

“Maize oil marker” means a marker in any one of the genomic amplicons of SEQ ID NO:1 through SEQ ID NO:186 and markers in linkage disequilibrium with a marker in said amplicons.

“Marker assay” means a method for detecting a polymorphism at a particular locus using a particular method, e.g., phenotype (such as seed color, flower color, or other visually detectable trait), restriction fragment length polymorphism (RFLP), single base extension, electrophoresis, sequence alignment, allelic specific oligonucleotide hybridization (ASO), RAPD, etc. Preferred marker assays include single base extension as disclosed in U.S. Pat. No. 6,013,431 and allelic discrimination where endonuclease activity releases a reporter dye from a hybridization probe as disclosed in U.S. Pat. No. 5,538,848, the disclosures of both of which are incorporated herein by reference.

“Linkage” is the lack of independent assortment of alleles at different loci into gametes during meiosis. For example, consider locus A with alleles “A” and “a” and locus B with alleles “B” and “b”. When a diploid formed from the union of gametes “AB” and “ab” undergoes meiosis, it can produce four types of gametes: “AB”, “Ab”, “aB” and “ab”. The null expectation is that there will be independent and equal segregation into each of the four possible genotypes, i.e., with no linkage, ¼ of the gametes will be of each genotype. Segregation of alleles into gametes at frequencies differing from h are attributed to linkage. Two loci are said to be “genetically linked” when they show this deviation from the expected equal frequency of ¼.

“Linkage disequilibrium” is defined in the context of the relative frequency of gamete types in a population of many individuals in a single generation. Again, consider locus A with alleles “A” and “a” and locus B with alleles “B” and “b,” if the frequency of allele A is p, a is p′, B is q and b is q′, then the expected frequency (with no linkage disequilibrium) of genotype AB is pq, Ab is pq′, aB is p′q and ab is p′q′. Any deviation from the expected frequency is called linkage disequilibrium. It is a nonrandom association of alleles at different loci.

“Quantitative Trait Locus (QTL)” means a locus that controls to some degree numerically representable traits that are usually continuously distributed.

“Haplotype” means the genotype for multiple loci or genetic markers in a haploid gamete. Generally, these loci or markers reside within a relatively small and defined region of a chromosome. A preferred haplotype comprises the 10 cM region or the 5 cM region or the 2 cM region surrounding an informative marker having a significant association with oil.

“Hybridizing” means the capacity of two nucleic acid molecules or fragments thereof to form anti-parallel, double-stranded nucleotide structure. The nucleic acid molecules of this invention are capable of hybridizing to other nucleic acid molecules under certain circumstances. A nucleic acid molecule is said to be the “complement” of another nucleic acid molecule if the molecules exhibit “complete complementarity,” i.e., each nucleotide in one sequence is complementary to its base pairing partner nucleotide in another sequence. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Nucleic acid molecules that hybridize to other nucleic acid molecules, e.g., at least under low stringency conditions are said to be “hybridizable cognates” of the other nucleic acid molecules. Conventional stringency conditions are described by Sambrook et al., Molecular Cloning, A Laboratory Manual, 2nd Ed., Cold Spring Harbor Press, Cold Spring Harbor, N.Y. (1989) and by Haymes et al., Nucleic Acid Hybridization, A Practical Approach, IRL Press, Washington, D.C. (1985), each of which is incorporated herein by reference. Departures from complete complementarity are therefore permissible, as long as such departures do not completely preclude the capacity of the molecules to form a double-stranded structure. Thus, in order for a nucleic acid molecule to serve as a primer or probe, it need only be sufficiently complementary in sequence to be able to form a stable double-stranded structure under the particular solvent and salt concentrations employed. Appropriate stringency conditions that promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6, incorporated herein by reference. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

“Sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components that are shared by the two aligned sequences divided by the total number of components in reference sequence segment, i.e., the entire reference sequence or a smaller defined part of the reference sequence. “Percent identity” is the identity fraction times 100. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc. Burlington, Mass.). Polynucleotides of the present invention that are variants of the polynucleotides provided herein will generally demonstrate significant identity with the polynucleotides provided herein. Of particular interest are polynucleotide homologs having at least about 70% sequence identity, at least about 80% sequence identity, at least about 90% sequence identity, and more preferably even greater, such as 98% or 99% sequence identity with polynucleotide sequences described herein.

“Genetic transformation” means a process of introducing a DNA construct (e.g., a vector or expression cassette) into a cell or protoplast in which that exogenous DNA is incorporated into a chromosome or is capable of autonomous replication.

“Exogenous gene” means a gene or partial gene that is not normally present in a given host genome in the exogenous gene's present form. In this respect, the gene itself may be native to the host genome; however, the exogenous gene will comprise the native gene altered by the addition or deletion of one or more different regulatory elements.

“Expression” means the combination of intracellular processes, including transcription and translation undergone by a coding DNA molecule such as a structural gene to produce a polypeptide.

“Progeny” means any subsequent generation, including the seeds and plants therefrom, that is derived from a particular parental plant or set of parental plants.

“Promoter” means a recognition site on a DNA sequence or group of DNA sequences that provides an expression control element for a structural gene and to which RNA polymerase specifically binds and initiates RNA synthesis (transcription) of that gene.

“R₀ transgenic plant” means a plant that has been directly transformed with a selected DNA or has been regenerated from a cell or cell cluster that has been transformed with a selected DNA.

“Regeneration” means the process of growing a plant from a plant cell (e.g., plant protoplast, callus or explant).

“DNA construct” means a chimeric DNA molecule that is designed for introduction into a host genome by genetic transformation. Preferred DNA constructs will comprise all of the genetic elements necessary to direct the expression of one or more exogenous genes. In particular embodiments of the instant invention, it may be desirable to introduce a DNA construct into a host cell in the form of an expression cassette.

“Transformed cell” means a cell the DNA complement of which has been altered by the introduction of an exogenous DNA molecule into that cell.

“Transgene” means a segment of DNA that has been incorporated into a host genome or is capable of autonomous replication in a host cell and is capable of causing the expression of one or more cellular products. Exemplary transgenes will provide the host cell, or plants regenerated therefrom, with a novel phenotype relative to the corresponding non-transformed cell or plant. Transgenes may be directly introduced into a plant by genetic transformation or may be inherited from a plant of any previous generation that was transformed with the DNA segment.

“Transgenic plant” means a plant or progeny plant of any subsequent generation derived therefrom, wherein the DNA of the plant or progeny thereof contains an introduced exogenous DNA segment not originally present in a non-transgenic plant of the same strain. The transgenic plant may additionally contain sequences that are native to the plant being transformed, but wherein the “exogenous” gene has been altered in order to alter the level or pattern of expression of the gene.

“Transit peptide” means a polypeptide sequence that is capable of directing a polypeptide to a particular organelle or other location within a cell.

“Vector” means a DNA molecule capable of replication in a host cell or to which another DNA segment can be operatively linked so as to bring about replication of the attached segment. A plasmid is an exemplary vector.

“Purified” refers to a nucleic acid molecule or polypeptide separated from substantially all other molecules normally associated with it in its native state. More preferably, a substantially purified molecule is the predominant species present in a preparation. A substantially purified molecule may be greater than 60% free or 75% free or 90% free or 95% free from the other molecules (exclusive of solvent) present in the natural mixture. The terms “isolated and purified” and “substantially purified” are not intended to encompass molecules present in their native state.

As used herein “yield” means the production of a crop, e.g., shelled corn kernels or soybean or cotton fiber, per unit of production area, e.g., in bushels per acre or metric tons per hectare, often reported on a moisture adjusted basis, e.g., corn is typically reported at 15.5% moisture. Moreover a bushel of corn is defined by law in the State of Iowa as 56 pounds by weight, a useful conversion factor for corn yield is 100 bushels per acre is equivalent to 6.272 metric tons per hectare. Other measurements for yield are in common practice.

The molecules and organisms of the invention may also be “recombinant,” which describes (a) nucleic acid molecules that are constructed or modified outside of cells and that can replicate or function in a living cell, (b) molecules that result from the transcription, replication or translation of recombinant nucleic acid molecules, or (c) organisms that contain recombinant nucleic acid molecules or are modified using recombinant nucleic acid molecules.

As used herein a “transgenic” organism, e.g., plant or seed, is one whose genome has been altered by the incorporation of exogenous genetic material or additional copies of native genetic material, e.g., by transformation or recombination of the organism or an ancestor organism. Transgenic plants include progeny plants of an original plant derived from a transformation process including progeny of breeding transgenic plants with wild type plants or other transgenic plants Crop plants of interest in the present invention include, but are not limited to, maize, soybean, cotton, wheat, sorghum, canola (oilseed rape), sunflower, safflower and flax.

“Enhanced protein activity” in a recombinant cell or organism is determined by reference to a wild-type plant or to the non-recombinant ancestor plant line or, in the case where the ancestor is a recombinant plant, to the parental line prior to the most recent recombinant insertion intended to promote the specific enhanced protein activity and can be determined by direct or indirect measurement. Direct measurement of protein activity might include an analytical assay for the protein, per se, or enzymatic product of protein activity. Indirect assay might include measurement of a property affected by the protein. Enhanced protein activity can be achieved by linking a constitutive promoter to the gene encoding the protein. Reduced protein activity can be achieved by a variety of mechanisms including anti-sense, co-suppression, double stranded RNA (dsRNA), mutation or knockout. Anti-sense, co-suppression and dsRNA mechanisms will reduce the level of protein expressed and the activity will be reduced as compared to wild-type expression levels. A mutation in the gene coding for a protein may not decrease the protein expression but instead interfere with the protein's function to cause reduced protein activity. A knockout can be achieved by homologous recombination with less than the whole gene.

As used herein “gene suppression” means any of the well-known methods for suppressing expression of protein from a gene including sense suppression, anti-sense suppression and RNAi suppression. In suppressing oil-associated genes to provide plants with reduced levels of seed oil, anti-sense and RNAi gene suppression methods are preferred. More particularly, for a description of anti-sense regulation of gene expression in plant cells see U.S. Pat. No. 5,107,065 and for a description of RNAi gene suppression in plants by transcription of a dsRNA see U.S. Pat. No. 6,506,559, U.S. Patent Application Publication No. 2002/0168707 A1, and U.S. patent application Ser. No. 09/423,143 (see WO 98/53083), Ser. No. 09/127,735 (see WO 99/53050) and Ser. No. 09/084,942 (see WO 99/61631), all of which are incorporated herein by reference. Suppression of an oil-associated gene by RNAi can be achieved using a recombinant DNA construct having a promoter operably linked to a DNA element comprising a sense and anti-sense element of a segment of genomic DNA of the oil-associated gene, e.g., a segment of at least about 23 nucleotides, more preferably about 50 to 200 nucleotides where the sense and anti-sense DNA components can be directly linked or joined by an intron or artificial DNA segment that can form a loop when the transcribed RNA hybridizes to form a hairpin structure. For example, genomic DNA from a polymorphic locus of SEQ ID NO:1 through SEQ ID NO:186 can be used in a recombinant construct for suppression of a cognate oil-associated gene by RNAi suppression.

Characteristics of Oil-Associated Genes

This invention provides nucleic acid molecules comprising DNA sequence representing oil-associated genes having a nucleic acid sequence of SEQ ID NO:187 through SEQ ID NO:345 or fragments of such oil-associated genes such as substantial parts of oil-associated genes providing the protein coding sequence part of the oil-associated gene. The oil-associated genes of this invention have been identified by marker trait association.

Homologous oil-associated genes have been identified in other plants and in other organisms such as fungi, algae and bacteria using the nucleic acid sequence of a known oil-associated gene or the amino acid sequence of a protein encoded by an oil-associated gene in any of a variety of search algorithms, e.g., the BLAST search algorithm, in public or proprietary DNA and protein databases. Existence of a gene is inferred if significant sequence similarity extends over the sequence of the target gene. Because homology-based methods may overlook genes unique to the source organism, for which homologous nucleic acid molecules have not yet been identified in databases, gene prediction programs are also used. Gene prediction programs generally use “signals” in the sequence, such as splice sites or “content” statistics, such as codon bias, to predict gene structures (Stormo, Genome Research 10: 394-397, 2000). Identified homologs of oil-associated genes are listed in Table 1.

With respect to nucleotide sequences, degeneracy of the genetic code provides the possibility to substitute at least one base of the base sequence of a gene with a different base without causing the amino acid sequence of the polypeptide produced from the gene to be changed. Hence, the DNA of the present invention may also have any codon changed in a sequence of SEQ ID NO: 1 through SEQ ID NO: 345 by substitution in accordance with degeneracy of genetic code. See U.S. Pat. No. 5,500,365, incorporated herein by reference.

More particularly, the homologous oil-associated genes can be characterized by reference to an artificial consensus sequence of conserved amino acids determined from an alignment of protein sequence encoded by such homologs.

Characteristics of Maize Oil Markers

The maize loci of this invention comprise a DNA sequence that comprises at least 20 consecutive nucleotides and includes or is adjacent to one or more polymorphisms identified in Table 1. Such maize loci have a nucleic acid sequence having at least 90% sequence identity or at least 95% or for some alleles at least 98% and in many cases at least 99% sequence identity, to the sequence of the same number of nucleotides in either strand of a segment of maize DNA that includes or is adjacent to the polymorphism. The nucleotide sequence of one strand of such a segment of maize DNA may be found in a polymorphic locus with a sequence in the group consisting of SEQ ID NO:1 through SEQ ID NO:186. It is understood by the very nature of polymorphisms that for at least some alleles there will be no identity to the polymorphism, per se. Thus, sequence identity can be determined for a sequence that is exclusive of the polymorphism sequence. The polymorphisms in each locus are identified more particularly in Table 1.

For many genotyping applications it is useful to employ as markers polymorphisms from more than one locus. Thus, aspects of the invention use a collection of different loci. The number of loci in such a collection can vary but will be a finite number, e.g., as few as 2 or 5 or 10 or 25 loci or more, for instance up to 40 or 75 or 100 or more loci.

Another aspect of the invention provides nucleic acid molecules that are capable of hybridizing to the polymorphic maize loci of this invention, e.g., PCR primers and hybridization probes. In certain embodiments of the invention, e.g., which provide PCR primers, such molecules comprise at least 15 nucleotide bases. Molecules useful as primers can hybridize under high stringency conditions to one of the strands of a segment of DNA in a polymorphic locus of this invention. Primers for amplifying DNA are provided in pairs, i.e., a forward primer and a reverse primer. One primer will be complementary to one strand of DNA in the locus and the other primer will be complementary to the other strand of DNA in the locus, i.e., the sequence of a primer is at least 90% or at least 95% identical to a sequence of the same number of nucleotides in one of the strands. It is understood that such primers can hybridize to a sequence in the locus that is distant from the polymorphism, e.g., at least 5, 10, 20, 50 or up to about 100 nucleotide bases away from the polymorphism. Design of a primer of this invention will depend on factors well known in the art, e.g., avoidance of repetitive sequence.

Another aspect of the nucleic acid molecules of this invention are hybridization probes for polymorphism assays. In one aspect of the invention such probes are oligonucleotides comprising at least 12 nucleotide bases and a detectable label. The purpose of such a molecule is to hybridize, e.g., under high stringency conditions, to one strand of DNA in a segment of nucleotide bases that includes or is adjacent to the polymorphism of interest in an amplified part of a polymorphic locus. Such oligonucleotides are at least 90% or at least 95% identical to the sequence of a segment of the same number of nucleotides in one strand of maize DNA in a polymorphic locus. The detectable label can be a radioactive element or a dye. In preferred aspects of the invention, the hybridization probe further comprises a fluorescent label and a quencher, e.g., for use in hybridization probe assays of the type known as Taqman assays, available from Applied Biosystems of Foster City, Calif.

For assays where the molecule is designed to hybridize adjacent to a polymorphism that is detected by single base extension, e.g., of a labeled dideoxynucleotide, such molecules can comprise at least 15 or at least 16 or 17 nucleotide bases in a sequence that is at least 90% or at least 95% identical to a sequence of the same number of consecutive nucleotides in either strand of a segment of polymorphic maize DNA. Oligonucleotides for single base extension assays are available from Orchid Bioystems.

Such primer and probe molecules are generally provided in groups of two primers and one or more probes for use in genotyping assays. Moreover, it is often desirable to conduct a plurality of genotyping assays for a plurality of polymorphisms. Thus, this invention also provides collections of nucleic acid molecules, e.g., in sets that characterize a plurality of polymorphisms.

Characteristics of Protein and Polypeptide Molecules

The nucleic acid molecules of this invention encode certain proteins or smaller polypeptide molecules including those having an amino acid sequence of SEQ ID NO: 346 through SEQ ID NO: 504. Homologs of the polypeptides of the present invention may be identified by comparison of the amino acid sequence of the polypeptide to amino acid sequences of polypeptides from the same or different plant sources, e.g., manually or by using known homology-based search algorithms such as those commonly known and referred to as BLAST, FASTA, and Smith-Waterman.

A further aspect of the invention comprises functional homolog proteins that differ in one or more amino acids from those of a polypeptide provided herein as the result of one or more of the well-known conservative amino acid substitutions, e.g., valine is a conservative substitute for alanine and threonine is a conservative substitute for serine. Conservative substitutions for an amino acid within the native polypeptide sequence can be selected from other members of a class to which the naturally occurring amino acid belongs. Representative amino acids within these various classes include, but are not limited to, (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral nonpolar (hydrophobic) amino acids such as alanine, leucine, isoleucine, valine, proline, phenylalanine, tryptophan, and methionine. Conserved substitutes for an amino acid within a native amino acid sequence can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Naturally conservative amino acids substitution groups are: valine-leucine, valine-isoleucine, phenylalanine-tyrosine, lysine-arginine, alanine-valine, aspartic acid-glutamic acid, and asparagine-glutamine. A further aspect of the invention comprises polypeptides that differ in one or more amino acids from those of a described protein sequence as the result of deletion or insertion of one or more amino acids in a native sequence.

Recombinant DNA Constructs for Plant Transformation

The present invention contemplates the use of polynucleotides that encode a protein effective for imparting altered oil levels in plants. Such polynucleotides are assembled in recombinant DNA constructs using methods known to those of ordinary skill in the art. A useful technology for building DNA constructs and vectors for transformation is the GATEWAY™ cloning technology (available from Invitrogen Life Technologies, Carlsbad, Calif.), which uses the site-specific recombinase LR cloning reaction of the Integrase/att system from bacteriophage lambda vector construction instead of restriction endonucleases and ligases. The LR cloning reaction is disclosed in U.S. Pat. Nos. 5,888,732 and 6,277,608, U.S. Patent Application Publications 2001283529, 2001282319 and 20020007051, all of which are incorporated herein by reference. The GATEWAY™ Cloning Technology Instruction Manual, which is supplied by Invitrogen, also provides concise directions for routine cloning of any desired DNA into a vector comprising operable plant expression elements.

Transgenic DNA constructs used for transforming plant cells will comprise the heterologous DNA that one desires to introduced into and a promoter to express the heterologous DNA in the host maize cells. As is well known in the art, such constructs typically also comprise a promoter and other regulatory elements, 3′ untranslated regions (such as polyadenylation sites), transit or signal peptides and marker genes elements as desired. For instance, see U.S. Pat. Nos. 5,858,642 and 5,322,938, which disclose versions of the constitutive promoter derived from cauliflower mosaic virus (CaMV35S), U.S. Pat. No. 6,437,217, which discloses a maize RS81 promoter, U.S. Pat. No. 5,641,876, which discloses a rice actin promoter, U.S. Pat. No. 6,426,446, which discloses a maize RS324 promoter, U.S. Pat. No. 6,429,362, which discloses a maize PR-1 promoter, U.S. Pat. No. 6,232,526, which discloses a maize A3 promoter, U.S. Pat. No. 6,177,611, which discloses constitutive maize promoters, U.S. Pat. No. 6,433,252, which discloses a maize L3 oleosin promoter, U.S. Pat. No. 6,429,357, which discloses a rice actin 2 promoter and intron, U.S. Pat. No. 5,837,848, which discloses a root specific promoter, U.S. Pat. No. 6,084,089, which discloses cold inducible promoters, U.S. Pat. No. 6,294,714, which discloses light inducible promoters, U.S. Pat. No. 6,140,078, which discloses salt inducible promoters, U.S. Pat. No. 6,252,138, which discloses pathogen inducible promoters, U.S. Pat. No. 6,175,060, which discloses phosphorus deficiency inducible promoters, U.S. Patent Application Publication 2002/0192813A1, which discloses 5′, 3′ and intron elements useful in the design of effective plant expression vectors, U.S. patent application Ser. No. 09/078,972, which discloses a coixin promoter, and U.S. patent application Ser. No. 09/757,089, which discloses a maize chloroplast aldolase promoter, all of which are incorporated herein by reference.

In many aspects of the invention it is preferred that the promoter element in the DNA construct should be seed or kernel-tissue specific. Such promoters can be identified and isolated by those skilled in the art from the regulatory region of plant genes that are over expressed in seed tissue, e.g., embryo or endosperm. For example, specific-seed tissue-specific promoters for use in this invention include an L3 oleosin promoter as disclosed in U.S. Pat. No. 6,433,252, a gamma coixin promoter as disclosed in U.S. patent application Ser. No. 09/078,972, and emb5 promoter as disclosed in U.S. provisional application Serial No. 60/434,242, all of which are incorporated herein by reference.

In general, it is preferred to introduce heterologous DNA randomly, i.e., at a non-specific location, in the plant genome. In special cases it may be useful to target heterologous DNA insertion in order to achieve site-specific integration, e.g., to replace an existing gene in the genome. In some other cases it may be useful to target a heterologous DNA integration into the genome at a predetermined site from which it is known that gene expression occurs. Several site-specific recombination systems exist that are known to function in plants and include cre-lox as disclosed in U.S. Pat. No. 4,959,317 and FLP-FRT as disclosed in U.S. Pat. No. 5,527,695, both incorporated herein by reference.

Constructs and vectors may also include a transit peptide for targeting of a gene target to a plant organelle, particularly to a chloroplast, leucoplast or other plastid organelle. For a description of the use of a chloroplast transit peptide see U.S. Pat. No. 5,188,642, incorporated herein by reference.

In practice, DNA is introduced into only a small percentage of target cells in any one experiment. Selectable marker genes are used to provide an efficient system for identification of those cells that are stably transformed by receiving and integrating a transgenic DNA construct into their genomes. Preferred selectable marker genes confer resistance to a selective agent, such as an antibiotic or herbicide. Potentially transformed cells are exposed to the selective agent. In the population of surviving cells will be those cells where, generally, the resistance-conferring gene has been integrated and expressed at sufficient levels to permit cell survival. Cells may be tested further to confirm stable integration of the exogenous DNA. Useful selectable marker genes include those conferring resistance to antibiotics such as kanamycin (nptII), hygromycin B (aph IV) and gentamycin (aac3 and aacC4) or resistance to herbicides such as glufosinate (bar or pat) and glyphosate (EPSPS). Examples of such selectable marker genes are illustrated in U.S. Pat. Nos. 5,550,318; 5,633,435; 5,780,708 and 6,118,047, all of which are incorporated herein by reference. Screenable markers that provide an ability to visually identify transformants can also be employed, e.g., a gene expressing a colored or fluorescent protein such as a luciferase or green fluorescent protein (GFP) or a gene expressing a beta-glucuronidase or uidA gene (GUS) for which various chromogenic substrates are known.

Exogenous Oil-Associated Genes for Modification of Plant Phenotypes

A particularly important advance of the present invention is that it provides DNA sequences useful for producing desirable oil-related phenotypes in plants, preferably in crop plants such as soybean, cotton, canola, sunflower, safflower, flax and most preferably in maize.

The choice of a selected DNA sequence for expression in a plant host cell in accordance with the invention will depend on the purpose of gene expression, e.g., expression of a native gene or homolog by a constitutive promoter, over expression of a native gene or homolog, suppression of a native gene, or altered tissue- or stage-specific expression of a native gene or homolog by a tissue- or stage-specific promoter.

In certain embodiments of the invention, transformation of a recipient cell may be carried out with more than one exogenous DNA coding region. As used herein, an “exogenous coding region” or “selected coding region” is a coding region not normally found in the host genome in an identical context. By this, it is meant that the coding region may be isolated from a different species than that of the host genome, or alternatively, isolated from the host genome, but it is operably linked to one or more regulatory regions that differ from those found in the unaltered, native gene. Two or more exogenous coding regions also can be supplied in a single transformation event using either distinct transgene-encoding vectors, or using a single vector incorporating two or more coding sequences.

Enhancement of an oil-related trait can also be effected by suppression of one or more genes that express proteins that divert oil producing materials into competing products or that degrade oil products. Site-directed inactivation of a gene, while possible, is typically difficult to achieve. Other more effective methods of gene suppression include the use anti-sense RNA, co-suppression, interfering RNA, processing defective RNA, transposon tagging, backcrossing or homologous recombination. Post transcriptional gene suppression by RNA interference is a superior and preferred method of gene suppression. In a preferred embodiment, gene suppression may complement over expression of an oil-associated gene.

Transformation Methods and Transgenic Plants

Methods and compositions for transforming plants by introducing a transgenic DNA construct into a plant genome in the practice of this invention can include any of the well-known and demonstrated methods. Preferred methods of plant transformation are microprojectile bombardment as illustrated in U.S. Pat. Nos. 5,015,580; 5,550,318; 5,538,880; 6,160,208: 6,194,636 and 6,399,861 and Agrobacterium-mediated transformation as illustrated in U.S. Pat. Nos. 5,824,877; 5,591,616; 5,981,840 and 6,384,301, all of which are incorporated herein by reference. See also U.S. application Ser. No. 09/823,676, incorporated herein by reference, for a description of vectors, transformation methods, and production of transformed Arabidopsis thaliana plants where genes in a recombinant DNA construct are constitutively expressed by a CaMV35S promoter.

Transformation methods of this invention to provide plants with enhanced oil traits are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Those cells that are capable of proliferating as callus also are recipient cells for genetic transformation. Practical transformation methods and materials for making transgenic plants of this invention, e.g., various media and recipient target cells, transformation of immature embryos and subsequent regeneration of fertile transgenic plants are disclosed in U.S. Pat. No. 6,194,636 and U.S. patent application Ser. No. 09/757,089, which are incorporated herein by reference.

Regeneration and Seed Production

Cells that survive the exposure to the selective agent, or cells that have been scored positive in a screening assay, may be cultured in media that supports regeneration of plants. Such media is well-known to one of skill in the art.

The transformed cells, identified by selection or screening and cultured in an appropriate medium that supports regeneration, will then be allowed to mature into plants. Developing plantlets are transferred to soil-less plant growth mix, and hardened off, e.g., in an environmentally controlled chamber at about 85% relative humidity, 600 ppm CO₂, and 25-250 microeinsteins m⁻² s⁻¹ of light, prior to transfer to a greenhouse or growth chamber for maturation. Plants are preferably matured either in a growth chamber or greenhouse. Plants are regenerated from about 6 wk to 10 months after a transformant is identified, depending on the initial tissue. During regeneration, cells are grown on solid media in tissue culture vessels. Regenerating plants are preferably grown at about 19° C. to 28° C. After the regenerating plants have reached the stage of shoot and root development, they may be transferred to a greenhouse-for further growth and testing. Plants may be pollinated using conventional plant breeding methods known-to those of skill in the art and seed produced.

Progeny may be recovered from transformed plants and tested for expression of the exogenous expressible gene. The transgenic seeds of this invention can be harvested from fertile transgenic plants and be used to grow progeny generations of transformed plants of this invention including hybrid plants; said progeny generations will contain the DNA construct expressing an oil-associated gene, which provides the benefits of enhanced oil production or storage

Seeds of R0 transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/L agarose. In embryo rescue, large embryos (defined as greater than 3 mm in length) are germinated directly on an appropriate media. Embryos smaller than that may be cultured for 1 wk on media containing the above ingredients along with 10⁻⁵M abscisic acid and then transferred to growth regulator-free medium for germination.

Characterization of Transgenic Plants for Presence of Exogenous DNA

To confirm the presence of the exogenous DNA in regenerating plants, a variety of assays may be performed. Such assays include, for example, “molecular biological” assays, such as Southern and Northern blotting and PCR; “biochemical” assays, such as detecting the presence of RNA, e.g., double-stranded RNA, or a protein product, e.g., by immunological means (ELISAs and Western blots) or by enzymatic function; plant part assays, such as leaf or root assays; and also, by analyzing the phenotype of the whole regenerated plant. Genomic DNA may be isolated from callus cell lines or any plant parts to determine the presence of the exogenous gene through the use of techniques well known to those skilled in the art.

The presence of DNA elements introduced through the methods of this invention may be determined by polymerase chain reaction (PCR). Using this technique, discreet fragments of DNA are amplified and detected by gel electrophoresis. This type of analysis permits one to determine whether a gene is present in a stable transformant, but it does not necessarily prove integration of the introduced gene into the host cell genome. Typically, DNA has been integrated into the genome of all transformants that demonstrate the presence of the gene through PCR analysis. In addition, it is not possible using PCR techniques to determine whether transformants have exogenous genes introduced into different sites in the genome, i.e., whether transformants are of independent origin. Using PCR techniques, it is possible to clone fragments of the host genomic DNA adjacent to an introduced gene.

Positive proof of DNA integration into the host genome and the independent identities of transformants may be determined using the technique of Southern hybridization. Using this technique, specific DNA sequences that were introduced into the host genome and flanking host DNA sequences can be identified. Hence the Southern hybridization pattern of a given transformant serves as an identifying characteristic of that transformant. In addition, it is possible through Southern hybridization to demonstrate the presence of introduced genes in high molecular weight DNA, i.e., confirm that the introduced gene has been integrated into the host cell genome. The technique of Southern hybridization provides information that can be obtained using PCR, e.g., the presence of a gene, but also demonstrates integration into the genome and characterizes each individual transformant. It is contemplated that using the techniques of dot or slot blot hybridization, which are modifications of Southern hybridization techniques, one could obtain the same information that is derived from PCR, e.g., the presence of a gene.

Both PCR and Southern hybridization techniques can be used to demonstrate transmission of a transgene to progeny. In most instances the characteristic Southern hybridization pattern for a given transformant will segregate in progeny as one or more Mendelian genes, indicating stable inheritance of the transgene.

Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species. It is further contemplated that TAQMAN® technology (Applied Biosystems, Foster City, Calif.) may be used to quantitate both DNA and RNA in a transgenic cell.

Although Southern blotting and PCR may be used to detect the gene(s) in question, they do not provide information as to whether the gene is being expressed. Expression may be evaluated by specifically identifying the protein products of the introduced genes or evaluating the phenotypic changes brought about by their expression. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification.

Event-Specific Transgene Assays

Southern blotting, PCR and RT-PCR techniques can be used to identify the presence or absence of a given transgene but, depending upon experimental design, may not specifically and uniquely identify identical or related transgene constructs located at different insertion points within the recipient genome. To more precisely characterize the presence of transgenic material in a transformed plant, one skilled in the art could identify the point of insertion of the transgene and, using the sequence of the recipient genome flanking the transgene, develop an assay that specifically and uniquely identifies a particular insertion event. Many methods can be used to determine the point of insertion such as, but not limited to, Genome Walker™ technology (CLONTECH, Palo Alto, Calif.), Vectorette™ technology (Sigma, St. Louis, Mo.), restriction site oligonucleotide PCR, uneven PCR, and generation of genomic DNA clones containing the transgene of interest in a vector such as, but not limited to, lambda phage.

Once the sequence of the genomic DNA directly adjacent to the transgenic insert on either or both sides has been determined, one skilled in the art can develop an assay to specifically and uniquely identify the insertion event. For example, two oligonucleotide primers can be designed, one wholly contained within the transgene and one wholly contained within the flanking sequence, that can be used together with the PCR technique to generate a PCR product unique to the inserted transgene. In one embodiment, the two oligonucleotide primers for use in PCR could be designed such that one primer is complementary to sequences in both the transgene and adjacent flanking sequence such that the primer spans the junction of the insertion site whereas the second primer could be homologous to sequences contained wholly within the transgene. In another embodiment, the two oligonucleotide primers for use in PCR could be designed such that one primer is complementary to sequences in both the transgene and adjacent flanking sequence such that the primer spans the junction of the insertion site whereas the second primer could be homologous to sequences contained wholly within the genomic sequence adjacent to the insertion site. Confirmation of the PCR reaction may be monitored by, but not limited to, size analysis on gel electrophoresis, sequence analysis, hybridization of the PCR product to a specific radiolabeled DNA or RNA probe or to a molecular beacon, or use of the primers in conjugation with a TAQMAN™ probe and technology (Applied Biosystems, Foster City, Calif.)

Site-Specific Integration or Excision of Transgenes

It is specifically contemplated by the inventors that one could employ techniques for the site-specific integration or excision of transformation constructs prepared in accordance with the instant invention. An advantage of site-specific integration or excision is that it can be used to overcome problems associated with conventional transformation techniques, in which transformation constructs typically randomly integrate into a host genome and multiple copies of a construct may integrate. Site-specific integration can be achieved in plants by means of homologous recombination as disclosed, for example, in U.S. Pat. Nos. 5,527,695 and 5,658,772, incorporated herein by reference.

Deletion of Sequences Located Within the Transgenic Insert

During the transformation process it is often necessary to include ancillary sequences, such as selectable marker or reporter genes, for tracking the presence or absence of a desired trait gene transformed into the plant on the DNA construct. Such ancillary sequences often do not contribute to the desired trait or characteristic conferred by the phenotypic trait gene. Homologous recombination is a method by which introduced sequences may be selectively deleted in transgenic plants.

Deletion of sequences by homologous recombination relies upon directly repeated DNA sequences positioned about the region to be excised so that the repeated DNA sequences direct excision utilizing native cellular recombination mechanisms. The first fertile transgenic plants are crossed to produce either hybrid or inbred progeny plants, and from those progeny plants, one or more second fertile transgenic plants are selected that contain a second DNA sequence that has been altered by recombination, preferably resulting in the deletion of the ancillary sequence. The first fertile plant can be either hemizygous or homozygous for the DNA sequence containing the directly repeated DNA that will drive the recombination event as disclosed in U.S. application Ser. No. 09/521,557, incorporated herein by reference.

Detecting Polymorphisms

Polymorphisms in DNA sequences can be detected by a variety of effective methods well known in the art including those methods disclosed in U.S. Pat. Nos. 5,468,613 and 5,217,863 by hybridization to allele-specific oligonucleotides; in U.S. Pat. Nos. 5,468,613 and 5,800,944 by probe ligation; in U.S. Pat. No. 5,616,464-by probe linking; and in U.S. Pat. Nos. 6,004,744; 6,013,431; 5,595,890; 5,762,876; and 5,945,283 by labeled base extension, all of which are incorporated herein by reference.

In another preferred method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter fluorescence, e.g., by Forster-type energy transfer. A PCR reaction is designed such that forward and reverse primers hybridize to specific sequences of the target DNA flanking a polymorphism. The hybridization probe hybridizes to polymorphism-containing sequence within the amplified PCR product. In the subsequent PCR cycle, DNA polymerase with 5′→3′ exonuclease activity cleaves the probe and separates the reporter dye from the quencher dye resulting in increased fluorescence of the reporter. A useful assay is available from Applied Biosystems as the Taqman® allele discrimination assay, which employs four synthetic oligonucleotides in a single reaction that concurrently amplifies the maize genomic DNA, discriminates between the alleles present, and directly provides a signal for discrimination and detection. Two of the four oligonucleotides serve as PCR primers and generate a PCR product encompassing the polymorphism to be detected. Two others are allele-specific fluorescence-resonance-energy-transfer (FRET) probes. FRET probes incorporate a fluorophore and a quencher molecule in close proximity so that the fluorescence of the fluorophore is quenched. The signal from a FRET probe is generated by degradation of the FRET oligonucleotide, so that the fluorophore is released from proximity to the quencher, and is thus able to emit light when excited at an appropriate wavelength. In the assay, two FRET probes bearing different fluorescent reporter dyes are used, where a unique dye is incorporated into an oligonucleotide that can anneal with high specificity to only one of the two alleles. Useful reporter dyes include 6-carboxy-4,7,2′,7′-tetrachlorofluorecein (TET), VIC (a dye from Applied Biosystems, Foster City, Calif.), and 6-carboxyfluorescein phosphoramidite (FAM). A useful quencher is 6-carboxy-N,N,N′N′-tetramethylrhodamine (TAMRA). Additionally, the 3′ end of each FRET probe is chemically blocked so that it cannot act as a PCR primer. During the assay, maize genomic DNA is added to a buffer containing the two PCR primers and two FRET probes. Also present is a third fluorophore used as a passive reference, e.g., rhodamine X (ROX), to aid in later normalization of the relevant fluorescence values (correcting for volumetric errors in reaction assembly). Amplification of the genomic DNA is initiated. During each cycle of the PCR, the FRET probes anneal in an allele-specific manner to the template DNA molecules. Annealed (but not non-annealed) FRET probes are degraded by TAQ DNA polymerase as the enzyme encounters the 5′ end of the annealed probe, thus releasing the fluorophore from proximity to its quencher. Following the PCR reaction, the fluorescence of each of the two fluorescers, as well as that of the passive reference, is determined fluorometrically. The normalized intensity of fluorescence for each of the two dyes will be proportional to the amounts of each allele initially present in the sample, and thus the genotype of the sample can be inferred.

To design primers and probes for the assay, the locus sequence is first masked to prevent design of any of the three primers to sites that match known maize repetitive elements (e.g., transposons) or are of very low sequence complexity (di- or tri-nucleotide repeat sequences). Design of primers to such repetitive elements will result in assays of low specificity, through amplification of multiple loci or annealing of the FRET probes to multiple sites.

PCR primers are designed (a) to have a length in the size range of 18 to 25 bases and matching sequences in the polymorphic locus, (b) to have a calculated melting temperature in the range of 57° C. to 60° C., e.g., corresponding to an optimal PCR annealing temperature of 52° C. to 55° C., (c) to produce a product that includes the polymorphic site and has a length in the size range of 75 to 250 base pairs. The PCR primers are preferably located on the locus so that the polymorphic site is at least one base away from the 3′ end of each PCR primer. The PCR primers must not contain regions that are extensively self- or inter-complementary.

FRET probes are designed to span the sequence of the polymorphic site, preferably with the polymorphism located in the 3′ most ⅔ of the oligonucleotide. In the preferred embodiment, the FRET probes will have incorporated at their 3′ end a chemical moiety referred to as the minor groove binder (MGB) that, when the probe is annealed to the template DNA, binds to the minor groove of the DNA, thus enhancing the stability of the probe-template complex. In addition the use of a non-fluorescing or “dark” quencher is employed. The probes should have a length in the range of 12 to 17 bases and, with the 3′ MGB, have a calculated melting temperature of 5° C. to 7° C. above that of the PCR primers. Probe design is disclosed in U.S. Pat. Nos. 5,538,848; 6,084,102; and 6,127,121.

Use Of Polymorphisms To Establish Marker/Trait Associations

The polymorphisms in the loci of this invention can be used in marker/trait associations that are inferred from statistical analysis of genotypes and phenotypes of the members of a population. These members may be individual organisms of, e.g., maize, families of closely related individuals, inbred lines, dihaploids or other groups of closely related individuals. Such maize groups are referred to as “lines”, indicating line of descent. The population may be descended from a single cross between two individuals or two lines (e.g., a mapping population) or it may consist of individuals with many lines of descent. Each individual or line is characterized by a single or average trait phenotype and by the genotypes at one or more marker loci.

Several types of statistical analysis can be used to infer marker/trait association from the phenotype/genotype data, but all are used is to detect markers, i.e., polymorphisms, for which alternative genotypes have significantly different average phenotypes. For example, if a given marker locus A has three alternative genotypes (AA, Aa and aa), and if those three classes of individuals have significantly different phenotypes, then one infers that locus A is associated with the trait. The significance of differences in phenotype may be tested by several types of standard statistical tests such as linear regression of marker genotypes on phenotype or analysis of variance (ANOVA). Commercially available statistical software packages commonly used to do this type of analysis include SAS Enterprise Miner (SAS Institute Inc., Cary, N.C.) and Splus (Insightful Corporation. Cambridge, Mass.).

Often the goal of an association study is not simply to detect marker/trait associations, but to estimate the location of genes affecting the trait directly (i.e., QTLs) relative to the marker locations. In a simple approach to this goal, one makes a comparison among marker loci of the magnitude of difference among alternative genotypes or the level of significance of that difference. Trait genes are inferred to be located nearest the marker(s) that have the greatest associated genotypic difference. In a more complex analysis, such as interval mapping (Lander and Botstein, Genetics 121:185-199, 1989), each of many positions along the genetic map (say at 1 cM intervals) is tested for the likelihood that a QTL is located at that position. The genotype/phenotype data are used to calculate for each test position a LOD score (log of likelihood ratio). When the LOD score exceeds a critical threshold value, there is significant evidence for the location of a QTL at that position on the genetic map (which will fall between two particular marker loci).

1. Linkage Disequilibrium Mapping and Association Studies

Another approach to determining trait gene location is to analyze trait-marker associations in a population within which individuals differ at both trait and marker loci. Certain marker alleles may be associated with certain trait locus alleles in this population due to population genetic process such as the unique origin of mutations, founder events, random drift and population structure. This association is referred to as linkage disequilibrium. In linkage disequilibrium mapping, one compares the trait values of individuals with different genotypes at a marker locus. Typically, a significant trait difference indicates close proximity between marker locus and one or more trait loci. If the marker density is appropriately high and the linkage disequilibrium occurs only between very closely linked sites on a chromosome, the location of trait loci can be very precise.

A specific type of linkage disequilibrium mapping is known as association studies. This approach makes use of markers within candidate genes, which are genes that are thought to be functionally involved in development of the trait because of information such as biochemistry, physiology, transcriptional profiling and reverse genetic experiments in model organisms. In association studies, markers within candidate genes are tested for association with trait variation. If linkage disequilibrium in the study population is restricted to very closely linked sites (i.e., within a gene or between adjacent genes), a positive association provides nearly conclusive evidence that the candidate gene is a trait gene.

2. Positional Cloning and Transgenic Applications

Traditional linkage mapping typically localizes a trait gene to an interval between two genetic markers (referred to as flanking markers). When this interval is relatively small (say less than 1 Mb), it becomes feasible to precisely identify the trait gene by a positional cloning procedure. A high marker density is required to narrow down the interval length sufficiently. This procedure requires a library of large insert genomic clones (such as a BAC library), where the inserts are pieces (usually 100-150 kb in length) of genomic DNA from the species of interest. The library is screened by probe hybridization or PCR to identify clones that contain the flanking marker sequences. Then a series of partially overlapping clones that connects the two flanking clones (a “contig”) is built up through physical mapping procedures. These procedures include fingerprinting, STS content mapping and sequence-tagged connector methodologies. Once the physical contig is constructed and sequenced, the sequence is searched for all transcriptional units. The transcriptional unit that corresponds to the trait gene can be determined by comparing sequences between mutant and wild type strains, by additional fine-scale genetic mapping, and/or by functional testing through plant transformation. Trait genes identified in this way become leads for transgenic product development. Similarly, trait genes identified by association studies with candidate genes become leads for transgenic product development.

3. Marker-Aided Breeding and Marker-Assisted Selection

When a trait gene has been localized in the vicinity of genetic markers, those markers can be used to select for improved values of the trait without the need for phenotypic analysis at each cycle of selection. In marker-aided breeding and marker-assisted selection, associations between trait genes and markers are established initially through genetic mapping analysis (as in sections 1 or 2 above). In the same process, one determines which marker alleles are linked to favorable trait gene alleles. Subsequently, marker alleles associated with favorable trait gene alleles are selected in the population. This procedure will improve the value of the trait provided that there is sufficiently close linkage between markers and trait genes. The degree of linkage required depends upon the number of generations of selection because, at each generation, there is opportunity for breakdown of the association through recombination.

4. Prediction of Crosses for New Inbred Line Development

The associations between specific marker alleles and favorable trait gene alleles also can be used to predict what types of progeny may segregate from a given cross. This prediction may allow selection of appropriate parents to generation populations from which new combinations of favorable trait gene alleles are assembled to produce a new inbred line. For example, if line A has marker alleles previously known to be associated with favorable trait alleles at loci 1, 20 and 31, while line B has marker alleles associated with favorable effects at loci 15, 27 and 29, then a new line could be developed by crossing A x B and selecting progeny that have favorable alleles at all 6 trait loci.

5. Hybrid Prediction

Commercial corn seed is produced by making hybrids between two elite inbred lines that belong to different “heterotic groups”. These groups are sufficiently distinct genetically that hybrids between them show high levels of heterosis or hybrid vigor (i.e., increased performance relative to the parental lines). By analyzing the marker constitution of good hybrids, one can identify sets of alleles at different loci in both male and female lines that combine well to produce heterosis. Understanding these patterns, and knowing the marker constitution of different inbred lines, can allow prediction of the level of heterosis between different pairs of lines. These predictions can narrow down the possibilities of which line(s) of opposite heterotic group should be used to test the performance of a new inbred line.

6. Identity by Descent

One theory of heterosis predicts that regions of identity by descent (IBD) between the male and female lines used to produce a hybrid will reduce hybrid performance. Identity by descent can be inferred from patterns of marker alleles in different lines. An identical string of markers at a series of adjacent loci may be considered identical by descent if it is unlikely to occur independently by chance. Analysis of marker fingerprints in male and female lines can identify regions of IBD. Knowledge of these regions can inform the choice of hybrid parents, because avoiding IBD in hybrids is likely to improve performance. This knowledge may also inform breeding programs in that crosses could be designed to produce pairs of inbred lines (one male and one female) that show little or no IBD.

A fingerprint of an inbred line is the combination of alleles at a set of marker loci. High density fingerprints can be used to establish and trace the identity of germplasm, which has utility in germplasm ownership protection.

Genetic markers are used to accelerate introgression of transgenes into new genetic backgrounds (i.e., into a diverse range of germplasm). Simple introgression involves crossing a transgenic line to an elite inbred line and then backcrossing the hybrid repeatedly to the elite (recurrent) parent, while selecting for maintenance of the transgene. Over multiple backcross generations, the genetic background of the original transgenic line is replaced gradually by the genetic background of the elite inbred through recombination and segregation. This process can be accelerated by selection on marker alleles that derive from the recurrent parent.

Use of Polymorphism Assay for Mapping a Library of DNA Clones

The polymorphisms and loci of this invention are useful for identifying and mapping DNA sequence of QTLs and genes linked to the polymorphisms. For instance, BAC or YAC clone libraries can be queried using polymorphisms linked to a trait to find a clone containing specific QTLs and genes associated with the trait. For instance, QTLs and genes in a plurality, e.g., hundreds or thousands, of large, multi-gene sequences can be identified by hybridization with an oligonucleotide probe that hybridizes to a mapped or linked polymorphism. Such hybridization screening can be improved by providing clone sequence in a high density array. The screening method is more preferably enhanced by employing a pooling strategy to significantly reduce the number of hybridizations required to identify a clone containing the polymorphism. When the polymorphisms are mapped, the screening effectively maps the clones.

For instance, in a case where thousands of clones are arranged in a defined array, e.g., in 96-well plates, the plates can be arbitrarily arranged in three-dimensionally, arrayed stacks of wells each comprising a unique DNA clone. The wells in each stack can be represented as discrete elements in a three dimensional array of rows, columns and plates. In one aspect of the invention, the number of stacks and plates in a stack are about equal to minimize the number of assays. The stacks of plates allow the construction of pools of cloned DNA.

For a three-dimensionally arrayed stack, pools of cloned DNA can be created for (a) all of the elements in each row, (b) all of the elements of each column, and (c) all of the elements of each plate. Hybridization screening of the pools with an oligonucleotide probe that hybridizes to a polymorphism unique to one of the clones will provide a positive indication for one column pool, one row pool and one plate pool, thereby indicating the well element containing the target clone.

In the case of multiple stacks, additional pools of all of the clone DNA in each stack allow indication of the stack having the row-column-plate coordinates of the target clone. For instance, a 4608 clone set can be disposed in 48 96-well plates. The 48 plates can be arranged in 8 sets of 6-plate stacks providing 6×12×8 three-dimensional arrays of elements, i.e., each stack comprises 6 stacks of 8 rows and 12 columns. For the entire clone set there are 36 pools, i.e., 6 stack pools, 8 row pools, 12 column pools and 8 stack pools. Thus, a maximum of 36 hybridization reactions is required to find the clone harboring QTLs or genes associated or linked to each mapped polymorphism.

Once a clone is identified, genes within that clone can be tested for whether they affect the trait by analysis of recombinants in a mapping population, further linkage disequilibrium analysis, and ultimately transgenic testing. Additional genes can be identified by finding additional clones overlapping the one containing the original polymorphism through contig building, as described above.

Breeding Plants of the Invention

In addition to direct transformation of a particular plant genotype with a construct prepared according to the current invention, transgenic plants may be made by crossing a plant having a construct of the invention to a second plant lacking the construct. For example, a selected coding region operably linked to a promoter can be introduced into a particular plant variety by crossing, without the need for ever directly transforming a plant of that given variety. Therefore, the current invention not only encompasses a plant directly regenerated from cells that have been transformed in accordance with the current invention, but also the progeny of such plants. As used herein the term “progeny” denotes the offspring of any generation of a parent plant prepared in accordance with the instant invention, wherein the progeny comprises a construct prepared in accordance with the invention. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:

(a) plant seeds of the first (starting line) and second (donor plant line that comprises a transgene of the invention) parent plants;

(b) grow the seeds of the first and second parent plants into plants that bear flowers;

(c) pollinate a flower from the first parent plant with pollen from the second parent plant; and

(d) harvest seeds produced on the parent plant bearing the fertilized flower.

Backcrossing is herein defined as the process including the steps of:

(a) crossing a plant of a first genotype containing a desired gene, DNA sequence or element to a plant of a second genotype lacking the desired gene, DNA sequence or element;

(b) selecting one or more progeny plants containing the desired gene, DNA sequence or element;

(c) crossing the progeny plant to a plant of the second genotype; and

(d) repeating steps (b) and (c) for the purpose of transferring the desired gene, DNA sequence or element from a plant of a first genotype to a plant of a second genotype.

Plant Breeding

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

Backcrossing can be used to improve a starting plant. Backcrossing transfers a specific desirable trait from one source to an inbred or other plant that lacks that trait. This can be accomplished, for example, by first crossing a superior inbred (A) (recurrent parent) to a donor inbred (non-recurrent parent), which carries the appropriate gene(s) for the trait in question, for example, a construct prepared in accordance with the current invention. The progeny of this cross first are selected in the resultant progeny for the desired trait to be transferred from the non-recurrent parent, then the selected progeny are mated back to the superior recurrent parent (A). After five or more backcross generations with selection for the desired trait, the progeny are hemizygous for loci controlling the characteristic being transferred but are like the superior parent for most or almost all other genes. The last backcross generation would be selfed to give progeny that are pure breeding for the gene(s) being transferred, i.e., one or more transformation events.

Therefore, through a series a breeding manipulations, a selected transgene may be moved from one line into an entirely different line without the need for further recombinant manipulation. Transgenes are valuable in that they typically behave genetically as any other gene and can be manipulated by breeding techniques in a manner identical to any other corn gene. Therefore, one may produce inbred plants that are true breeding for one or more transgenes. By crossing different inbred plants, one may produce a large number of different hybrids with different combinations of transgenes. In this way, plants may be produced that have the desirable agronomic properties frequently associated with hybrids (“hybrid vigor”), as well as the desirable characteristics imparted by one or more transgene(s).

It is desirable to introgress the genes of the present invention into maize hybrids for characterization of the phenotype conferred by each gene in a transformed plant. The host genotype into which the transgene was introduced, preferably LH59, is an elite inbred and therefore only limited breeding is necessary in order to produce high yielding maize hybrids. The transformed plant, regenerated from callus is crossed, to the same genotype, e.g., LH59. The progeny are self-pollinated twice, and plants homozygous for the transgene are identified. Homozygous transgenic plants are crossed to a testcross parent in order to produce hybrids. The test cross parent is an inbred belonging to a heterotic group that is different from that of the transgenic parent and for which it is known that high yielding hybrids can be generated, for example hybrids are produced from crosses of LH59 to either LH 195 or LH200.

The following examples illustrate the identification of polymorphic markers useful for mapping and isolating genes of this invention and as markers of QTLs and genes associated with an oil-related trait. Other examples illustrate the identification of oil-related genes and partial genes. Still other examples illustrate methods for inserting genes of this invention into a plant expression vector, i.e., operably linked to a promoter and other regulatory elements, to confer an oil-related trait to a transgenic plant.

### EXAMPLE 1

This example illustrates the identification of oil-associated genes and maize oil markers.

a. Candidate Oil Genes

A set of more than 800 candidate oil genes was identified (a) as homologs of plant genes that are believed to be in an oil-related metabolic pathway of a model plant such as Arabidopsis thaliana; (b) by comparing transcription profiling results for high oil and low oil maize lines; and (c) by subtractive hybridization between endosperm tissues of high oil and low oil maize lines. The sequences of the candidate oil genes were queried against a proprietary collection of maize genes and partial maize genes, e.g., genomic sequence or ESTs, to identify a set of more than 800 candidate maize oil genes.

b. Maize Polymorphisms

Maize polymorphisms were identified by comparing alignments of DNA sequences from separate maize lines. Candidate polymorphisms were qualified by the following parameters:

(a) The minimum length of sequence for a synthetic reference sequence is 200 bases.

(b) The percentage identity of observed bases in a region of 15 bases on each side of a candidate SNP is 75%.

(c) The minimum phred quality in each of the various sequences at a polymorphism site is 35.

(d) The minimum phred quality in a region of 15 bases on each side of the polymorphism site is 20.

c. Oil Informative Markers

The SNP and Indel polymorphisms in each locus were qualified for detection by development of an assay, e.g., Taqman® allele discrimination assay (Applied Biosystems, Foster City, Calif.). Assay qualified polymorphisms are evaluated for oil informativeness by comparing allelic frequencies in the two parental lines of an association study population. The parent lines were representatives of an oil rich maize population and an oil poor maize population, i.e., the University of Illinois High Oil and Low Oil maize lines as described by Dudley and Lambert (1992, Maydica 37: 81-87). Informativeness is reported as an allelic frequency difference between parental populations, i.e., the high oil line and the low oil line. When one of the parents, e.g., the high oil line, is fixed, its allelic frequency is 1. Markers were qualified if they had an allelic frequency difference of at least 0.6. If the marker was fixed in either parent with a frequency of 0 or 1, a marker could be selected at a lower allelic frequency difference of at least 0.4. The informative markers were viewed on a genetic map to identify marker-deficient regions of chromosomes. Markers with lower allelic frequency difference, e.g., as low as 0.15, were selected to fill in the marker-deficient regions of chromosomes. A set of informative markers were used in a marker-trait association study to verify oil-associated genes from the set of candidate oil genes.

d. Labeled Probe Degradation Assay for SNP Detection

A quantity of maize genomic template DNA (e.g., about 2-20 ng) is mixed in 5 μL total volume with four oligonucleotides, which can be designed by Applied Biosystems, i.e., a forward primer, a reverse primer, a hybridization probe having a VIC reporter attached to the 5′ end, and a hybridization probe having a FAM reporter attached to the 5′ end as well as PCR reaction buffer containing the passive reference dye ROX. The PCR reaction is conducted for 35 cycles using a 60° C. annealing-extension temperature. Following the reaction, the fluorescence of each fluorophore as well as that of the passive reference is determined in a fluorimeter. The fluorescence value for each fluorophore is normalized to the fluorescence value of the passive reference. The normalized values are plotted against each other for each sample. The data points should fall into clearly separable clusters.

To confirm that an assay produces accurate results, each new assay is performed on a number of replicates of samples of known genotypic identity representing each of the three possible genotypes, i.e., two homozygous alleles and a heterozygous sample. To be a valid and useful assay, it must produce clearly separable clusters of data points, such that one of the three genotypes can be assigned for at least 90% of the data points, and the assignment is observed to be correct for at least 98% of the data points. Subsequent to this validation step, the assay is applied to progeny of a cross between two highly inbred individuals to obtain segregation data, which are then used to calculate a genetic map position for the polymorphic locus.

e. Marker Mapping

The maize markers were genetically mapped based on the genotypes of certain SNPs. The genotypes were combined with genotypes for public core SSR and RFLP markers scored on recombinant inbred lines. Before mapping, any loci showing distorted segregation (P<0.0 for a Chi-square test of a 1:1 segregation ratio) were removed. These loci could be added to the map later but without allowing them to change marker order.

A map was constructed using the JoinMap version 2.0 software, which is described by Stam (“Construction of integrated genetic linkage maps by means of a new computer package: JoinMap, The Plant Journal, 3: 739-744 (1993); Stam, P. and van Ooijen, J. W. “JoinMap version 2.0: Software for the calculation of genetic linkage maps (1995) CPRO-DLO, Wageningen). JoinMap implements a weighted-least squares approach to multipoint mapping in which information from all pairs of linked loci (adjacent or not) is incorporated. Linkage groups were formed using a LOD threshold of 5.0. The SSR and RFLP public markers were used to assign linkage groups to chromosomes. Linkage groups were merged within chromosomes before map construction.

Haldane's mapping function was used to convert recombination fractions to map distances. Lenient criteria was applied for excluding pairwise linkage data; only data with a LOD not greater than 0.001 or a recombination fraction not less than 0.499 are excluded. Parameters for ordering loci were a jump threshold of 5.0, a triplet threshold of 7.0 and a ripple value of 3. About 38% of the loci were ordered in two rounds of map construction with a jump threshold of 5.0, which prevents the addition of a locus to the map if such addition results in a jump of more than 5.0 to a goodness-of-fit criterion. The remaining loci were added to the map without application of such a jump threshold. Addition of these loci had a negligible effect on the map order and distances for the initial loci. Mapped SNP polymorphisms are identified in Table 2.

f. Marker Trait Association

The informative maize markers were used in an association study to identify which of the candidate genes were more significantly associated with oil level in corn (Zea mays).

The University of Illinois has corn lines differing in seed oil that have been developed by long-term selection. A high oil line (IHO) produces about 18% seed oil and a low oil line (ILO) produces about 1.5% seed oil. The IHO and ILO lines are available from the University of Illinois for research. A random mated population (RMn) was produced from random mating offspring of a cross between IHO and ILO by chain crossing for 10 generations to produce an RM10 population. From the RM10 population, 504 S1-derived lines were developed by selfing and these lines constitute an association study population. This population along with 72 control samples were genotyped using oil informative SNPs.

Phenotypes were measured on 504 association population lines in replicated field trials with an alph(0,1) incomplete block design. The field trials comprised the 504 lines grown in each of two years at each of 3 locations with 2 replicates per location. The lines were blocked within each replicate. These field trials were performed on the 504 RM10:S1 lines, per se, and on hybrids made by crossing each line to a tester line, i.e., line 7051.

Association was analyzed between the SNP markers and oil level in the RM10:S1 lines, per se, and in the hybrids. A mixed model analysis of variance was performed with sources of variation: location, reps within location, blocks and lines. Line effects for both % oil in the kernel and weight of oil per 200 kernels estimated from this model were used in a linear model analysis of variance based on single marker genotypes classes (AA, Aa and aa) for all genotyped markers. Through this analysis, a total of 186 markers showed a significant effect on the trait at the p<0.05 level. These 186 significant markers which are very likely to either reside within an oil gene or to be closely linked to an oil gene are in the 186 polymorphic loci of SEQ ID NO: 1 through SEQ ID NO:186 and identified more particularly in Table 1. A set of 159 of the candidate genes having sequence that either overlaps with, or is associated by linkage disequilibrium with, any one or more of the 186 genomic amplicons of SEQ ID NO:1 through SEQ ID NO:186 were identified and designated as oil-associated genes and are identified as having a cDNA sequence of SEQ ID NO:187 through SEQ ID NO:345. Because these oil-associated genes contain or are associated by linkage disequilibrium to a statistically significant maize oil marker, these oil-associated genes are most likely to be oil genes.

Table 1 provides a description of 186 genomic amplicons defining polymorphic loci of the maize oil markers of this invention, 159 oil-associated genes and the cognate proteins, and homologous protein sequence from other species (identified as disclosed in Example 3 below). These particular aspects of the invention are identified by:

SEQ_NUM, which refers to the sequence number of the nucleic acid sequence or amino acid sequence, e.g., a SEQ ID NO.______; and

SEQ_ID, which refers to an arbitrary identifying name for an amplicon, e.g. “nnn”, for an oil-associated gene, e.g., “MRT4577_nnnnC”, for a cognate protein of an oil-associated gene, e.g., “MRT4577_nnnnP”, of for a cognate protein of a homolog to an oil-associated gene, e.g., “MRT4577_nnnnP” or a name from a database such as GenBank, e.g., “gi:6539874”.

More particularly, the maize oil markers in the 186 genomic amplicons are described by:

MUTATION_ID, which refers to one or more arbitrary identifying names for each polymorphism;

START_POS, which refers to the position in the nucleotide sequence of the polymorphic maize DNA locus where the polymorphism begins;

END_POS, which refers to the position in the nucleotide sequence of the polymorphic maize DNA locus where the polymorphism ends; for SNPs the START_POS and END_POS are common;

TYPE, which refers to the identification of the polymorphism as an SNP or IND (Indel);

ALLELEn and STRAINn, which refer to the nucleotide sequence of a polymorphism in a specific allelic maize variety; and

GENE_ID refers to the SEQ_ID of the oil-associated gene identified later in Table 1 except in the case of amplicons of SEQ ID NO:162 through SEQ ID NO:186 where “unknown” indicates informative maize oil markers that are not associated with an identified oil-associated gene.

More particularly, the oil-associated genes and their cognate proteins are described by:

DESCRIPTION, which refers to a functional description of an oil-associated gene, e.g., “gene encoding MRT4577_nnnnP” or a functional description of a cognate protein, e.g., a GenBank annotation or “long ORF” indicating no known protein function for an amino acid sequence that is translated from a longest available ORF.

And, more particularly, the homologs of the oil-associated genes beginning with SEQ ID NO:505 are described by:

SPECIES, which refers to the species of origin for the DNA encoding the protein sequence of the homolog; and

HOMOLOG_BASE_PROTEIN, which refers to an arbitrary identifying name for the cognate protein of an oil-associated gene, e.g., “MRT4577_nnnnP” that provided the amino acid sequence that was used to identify the homolog of the oil-associated gene.

Table 2 provides genetic map positions of maize oil markers and linked oil-associated genes; a description of the probability of significance of the marker/trait association (as determined from per se or hybrid association analysis for the marker); and the identification and sequence number of the oil-associated gene and their translated proteins. More particularly, Table 2 identifies maize oil markers, oil-associated genes and proteins by

Map Position, which identifies the distance measured in cM from the 5′ end of a maize chromosome for the SNP identified by Mutation ID, which refers to an arbitrary identifying name for each polymorphism;

Seq Num, which refers to the sequence number of a genomic amplicon containing the maize oil marker;

Protein Seq Num, which refers to the sequence number of the amino acid sequence, e.g., a SEQ ID NO, for the cognate protein encoded by a linked oil-associated gene;

Pval % Oil Per se, which refers to probability of a test of significance of the regression of marker genotype on oil level as percent oil per kernel for inbred lines;

Pval % Oil Hybrid, which refers to probability of a test of significance of the regression of marker genotype on oil level as percent oil per kernel for hybrid lines;

Pval Oil/Kernel Per se, which refers to probability of a test of significance of the regression of marker genotype on oil level as oil weight per 200 kernels for inbred lines;

Pval Oil/Kernel Hybrid, which refers to probability of a test of significance of the regression of marker genotype on oil level as oil weight per 200 kernels for hybrid lines.

TABLE 2

| | | | Protein | Pval | Pval | Pval | Pval |
|---|---|---|---|---|---|---|---|
| Map | | Seq | Seq | % Oil | % Oil | Oil/Kernel | Oil/Kernel |
| Position | Mutation_ID | Num | Num | Per se | Hybrid | Per se | Hybrid |
| 1-3.7 | 111829 | 185 | — | 0.706 | 0.234 | 0.336 | 0.046 |
| 1-25.1 | 43230 | 80 | 425 | 0.030 | 0.228 | 0.042 | 0.037 |
| 1-44 | 104827 | 105 | 450 | 0.094 | 0.801 | 0.018 | 0.909 |
| 1-45 | 151360 | 156 | 499 | 0.025 | 0.811 | 0.005 | 0.395 |
| 1-46.8 | 37716 | 69 | 414 | 0.009 | 0.113 | 0.024 | 0.351 |
| 1-53.3 | 42173 | 79 | 424 | 0.020 | 0.050 | 0.024 | 0.907 |
| 1-58.4 | 116 | 1 | 346 | 0.059 | 0.018 | 0.018 | 0.395 |
| 1-60.3 | 143100 | 136 | 481 | 0.722 | 0.029 | 0.878 | 0.501 |
| 1-60.6 | 33819 | 57 | 402 | 0.200 | 0.039 | 0.043 | 0.640 |
| 1-60.6 | 40189 | 75 | 420 | 0.007 | 1.6E−4 | 0.062 | 0.172 |
| 1-83.2 | 34205 | 58 | 403 | 0.026 | 0.151 | 0.090 | 0.022 |
| 1-86.3 | 8984 | 14 | 359 | 0.405 | 8.0E−4 | 0.433 | 0.069 |
| 1-86.3 | 36286 | 62 | 407 | 0.261 | 7.3E−4 | 0.328 | 0.069 |
| 1-88.8 | 29829 | 42 | 387 | 0.063 | 0.164 | 0.597 | 0.029 |
| 1-88.8 | 37068 | 65 | 410 | 0.026 | 0.317 | 0.068 | 0.051 |
| 1-90.5 | 111828 | 133 | 478 | 0.052 | 0.198 | 0.018 | 0.014 |
| 1-91 | 113263 | 135 | 480 | 0.281 | 0.004 | 0.078 | 0.489 |
| 1-91.8 | 104474 | 104 | 449 | 0.047 | 0.346 | 0.776 | 0.069 |
| 1-96.9 | 36448 | 63 | 408 | 0.006 | 0.114 | 0.002 | 0.052 |
| 1-99 | 40655 | 77 | 422 | 0.029 | 0.272 | 0.052 | 0.080 |
| 1-99 | 107077 | 113 | 458 | 9.7E−6 | 0.014 | 9.1E−4 | 0.021 |
| 1-103.3 | 8719 | 10 | 355 | 0.167 | 0.728 | 0.008 | 0.271 |
| 1-124.6 | 33373 | 56 | 401 | 0.029 | 0.240 | 0.201 | 0.714 |
| 1-130.3 | 69565 | 93 | 438 | 0.032 | 0.201 | 0.568 | 0.962 |
| 1-165.6 | 108862 | 120 | 465 | 0.011 | 0.001 | 0.402 | 0.347 |
| 1-178.6 | 151382 | 154 | 497 | 0.027 | 0.480 | 0.116 | 0.509 |
| 1-200.3 | 30840 | 47 | 392 | 0.662 | 0.050 | 0.716 | 0.012 |
| 2-5.8 | 31064 | 48 | 393 | 0.091 | 0.002 | 0.143 | 0.064 |
| 2-12.9 | 104447 | 180 | — | 0.077 | 0.012 | 0.697 | 0.459 |
| 2-14.1 | 39289 | 73 | 418 | 0.095 | 0.016 | 0.778 | 0.571 |
| 2-17.5 | 106678 | 110 | 455 | 0.048 | 0.003 | 0.043 | 0.040 |
| 2-19.5 | 82235 | 99 | 444 | 0.018 | 0.002 | 0.045 | 0.009 |
| 2-33.9 | 80031 | 97 | 442 | 0.101 | 0.046 | 0.557 | 0.036 |
| 2-35.9 | 13691 | 22 | 367 | 0.225 | 0.469 | 0.040 | 0.419 |
| 2-78.2 | 11466 | 19 | 364 | 0.096 | 0.761 | 0.045 | 0.225 |
| 2-78.2 | 79073 | 96 | 441 | 0.020 | 0.825 | 0.015 | 0.413 |
| 2-78.2 | 108493 | 119 | 464 | 0.142 | 0.045 | 0.713 | 0.299 |
| 2-92.5 | 3177 | 4 | 349 | 0.082 | 0.334 | 0.038 | 0.224 |
| 2-92.9 | 84829 | 102 | 447 | 0.298 | 0.324 | 0.111 | 0.031 |
| 2-99.7 | 151288 | 155 | 498 | 0.549 | 0.036 | 0.245 | 0.846 |
| 2-106 | 111475 | 131 | 476 | 0.238 | 0.013 | 0.320 | 0.685 |
| 2-106.2 | 108013 | 117 | 462 | 0.574 | 0.033 | 0.441 | 0.591 |
| 2-107.6 | 2307 | 3 | 348 | 0.497 | 0.019 | 0.437 | 0.413 |
| 2-114.9 | 22775 | 34 | 379 | 0.036 | 0.064 | 0.424 | 0.160 |
| 2-123.4 | 104954 | 107 | 452 | 0.049 | 0.058 | 0.573 | 0.765 |
| 2-152.4 | 43579 | 81 | 426 | 0.064 | 0.123 | 0.037 | 0.659 |
| 2-164.2 | 735 | 2 | 347 | 0.497 | 0.920 | 0.048 | 0.729 |
| 2-164.2 | 76792 | 95 | 440 | 0.939 | 0.524 | 0.044 | 0.345 |
| 3-6 | 8911 | 165 | — | 0.067 | 0.561 | 0.045 | 0.979 |
| 3-6 | 51614 | 86 | 431 | 0.071 | 0.551 | 0.030 | 0.980 |
| 3-9.1 | 10667 | 18 | 363 | 0.009 | 0.193 | 0.068 | 0.262 |
| 3-19.7 | 19963 | 169 | — | 0.115 | 0.084 | 0.029 | 0.373 |
| 3-19.7 | 32137 | 53 | 398 | 2.4E−4 | 1.1E−4 | 2.3E−4 | 0.037 |
| 3-46.2 | 49293 | 84 | 429 | 0.036 | 0.003 | 0.167 | 0.030 |
| 3-52.3 | 109315 | 121 | 466 | 0.175 | 7.7E−4 | 0.527 | 0.040 |
| 3-53.5 | 25000 | 172 | — | 0.098 | 4.5E−4 | 0.350 | 0.157 |
| 3-54.1 | 21154 | 31 | 376 | 0.060 | 7.7E−4 | 0.543 | 0.542 |
| 3-54.1 | 109722 | 126 | 471 | 0.482 | 0.022 | 0.526 | 0.284 |
| 3-57.2 | 109509 | 124 | 469 | 0.394 | 0.006 | 0.464 | 0.213 |
| 3-58.6 | 29867 | 43 | 388 | 0.036 | 1.8E−8 | 0.696 | 0.169 |
| 3-59.3 | 4599 | 8 | 353 | 0.093 | 7.9E−4 | 0.562 | 0.571 |
| 3-59.3 | 21190 | 33 | 378 | 0.020 | 0.006 | 0.637 | 0.215 |
| 3-59.3 | 28923 | 39 | 384 | 0.150 | 9.6E−4 | 0.703 | 0.351 |
| 3-59.3 | 147511 | 149 | 493 | 0.116 | 0.001 | 0.588 | 0.571 |
| 3-59.3 | 147768 | 150 | 493 | 0.066 | 7.6E−4 | 0.627 | 0.524 |
| 3-60.4 | 8685 | 164 | — | 0.592 | 0.001 | 0.913 | 0.681 |
| 3-61 | 16729 | 26 | 371 | 0.229 | 0.112 | 0.198 | 0.005 |
| 3-61.7 | 32247 | 54 | 399 | 0.115 | 3.5E−4 | 0.891 | 0.113 |
| 3-62.7 | 9144 | 167 | — | 0.066 | 0.003 | 0.277 | 0.014 |
| 3-62.7 | 9739 | 16 | 361 | 0.031 | 0.003 | 0.439 | 0.130 |
| 3-111.4 | 110780 | 129 | 474 | 0.246 | 0.040 | 0.572 | 0.207 |
| 3-123.8 | 143969 | 140 | 485 | 0.015 | 0.158 | 0.081 | 0.438 |
| 3-127.7 | 9079 | 166 | — | 0.040 | 0.071 | 0.296 | 0.134 |
| 4-38.7 | 110069 | 182 | — | 0.026 | 0.048 | 0.188 | 0.108 |
| 4-38.7 | 111464 | 184 | — | 0.029 | 0.053 | 0.129 | 0.096 |
| 4-52.8 | 24647 | 36 | 381 | 0.013 | 0.084 | 0.382 | 0.827 |
| 4-53.2 | 156243 | 161 | 504 | 0.004 | 0.007 | 0.096 | 0.368 |
| 4-62.1 | 10671 | 20 | 365 | 0.156 | 0.040 | 0.337 | 0.099 |
| 4-64.9 | 38852 | 176 | — | 0.285 | 0.072 | 0.342 | 0.007 |
| 4-69.5 | 5021 | 9 | 354 | 0.341 | 0.499 | 0.098 | 0.004 |
| 4-69.5 | 37503 | 66 | 411 | 0.262 | 0.126 | 0.303 | 0.002 |
| 4-69.9 | 107276 | 114 | 459 | 0.006 | 0.331 | 0.017 | 0.016 |
| 4-71.4 | 84527 | 101 | 446 | 0.346 | 0.014 | 0.363 | 0.040 |
| 4-80 | 106845 | 112 | 457 | 0.112 | 0.042 | 0.393 | 0.434 |
| 4-107.7 | 106491 | 109 | 454 | 0.020 | 0.040 | 0.409 | 0.521 |
| 4-112.4 | 54460 | 87 | 432 | 0.037 | 0.146 | 0.124 | 0.150 |
| 4-122.4 | 151472 | 153 | 496 | 0.186 | 0.994 | 0.011 | 0.967 |
| 4-128.1 | 32049 | 52 | 397 | 0.195 | 0.620 | 0.756 | 0.011 |
| 4-135.8 | 17900 | 28 | 373 | 4.2E−4 | 0.037 | 3.7E−4 | 0.019 |
| 4-136.4 | 147219 | 148 | 492 | 0.038 | 0.214 | 0.104 | 0.029 |
| 5-1.6 | 24265 | 35 | 380 | 0.082 | 0.035 | 0.472 | 0.010 |
| 5-39.9 | 109403 | 123 | 468 | 2.0E−6 | 9.1E−5 | 0.135 | 0.006 |
| 5-41.7 | 16527 | 25 | 370 | 0.028 | 0.161 | 0.333 | 0.791 |
| 5-50.9 | 109342 | 122 | 467 | 0.005 | 0.167 | 0.015 | 0.024 |
| 5-51.9 | 16762 | 27 | 372 | 7.6E−5 | 0.018 | 4.9E−4 | 0.029 |
| 5-51.9 | 16767 | 168 | — | 1.2E−4 | 0.017 | 5.1E−4 | 0.033 |
| 5-62.3 | 51419 | 85 | 430 | 0.046 | 0.002 | 0.163 | 0.031 |
| 5-63 | 32272 | 55 | 400 | 5.7E−5 | 0.001 | 0.008 | 0.100 |
| 5-66.9 | 30000 | 174 | — | 0.004 | 6.5E−4 | 0.035 | 0.002 |
| 5-66.9 | 146415 | 147 | 491 | 1.1E−4 | 1.9E−5 | 0.163 | 0.037 |
| 5-69.6 | 144731 | 186 | — | 8.5E−4 | 2.8E−4 | 0.162 | 0.042 |
| 5-70.5 | 105854 | 181 | — | 0.205 | 0.011 | 0.976 | 0.063 |
| 5-71.7 | 143216 | 137 | 482 | 0.023 | 0.014 | 0.065 | 0.098 |
| 5-76.4 | 29820 | 41 | 386 | 0.010 | 5.8E−4 | 0.128 | 0.140 |
| 5-80.2 | 36637 | 64 | 409 | 0.020 | 0.052 | 0.087 | 0.365 |
| 5-104.5 | 58375 | 89 | 434 | 0.028 | 0.097 | 0.024 | 0.003 |
| 5-150.5 | 31084 | 49 | 394 | 0.025 | 0.210 | 0.350 | 0.763 |
| 6-17.3 | 154854 | 159 | 502 | 0.010 | 0.222 | 0.049 | 0.688 |
| 6-30.8 | 69630 | 94 | 439 | 0.484 | 0.678 | 0.094 | 0.047 |
| 6-37.3 | 36067 | 61 | 406 | 0.018 | 0.290 | 0.215 | 0.874 |
| 6-37.3 | 36073 | 61 | 406 | 0.014 | 0.165 | 0.234 | 0.945 |
| 6-43.1 | 30176 | 45 | 390 | 0.827 | 0.323 | 0.969 | 0.015 |
| 6-52.8 | 4463 | 7 | 352 | 3.9E−9 | 1.8E−12 | 2.3E−6 | 3.7E−9 |
| 6-53.1 | 60751 | 91 | 436 | 3.9E−9 | 1.6E−6 | 5.4E−7 | 2.2E−4 |
| 6-53.5 | 32034 | 51 | 396 | 5.0E−7 | 3.7E−4 | 5.3E−5 | 0.048 |
| 6-53.5 | 57758 | 88 | 433 | 6.3E−5 | 0.008 | 0.002 | 0.566 |
| 6-53.5 | 108212 | 118 | 463 | 8.6E−7 | 6.0E−4 | 4.0E−5 | 0.043 |
| 6-58.1 | 59008 | 90 | 435 | 9.7E−5 | 3.9E−4 | 8.2E−5 | 0.002 |
| 6-58.1 | 146195 | 146 | 490 | 5.3E−4 | 8.5E−4 | 1.8E−5 | 0.004 |
| 6-59.9 | 3277 | 5 | 350 | 0.004 | 0.215 | 0.087 | 0.515 |
| 6-59.9 | 105586 | 108 | 453 | 0.003 | 4.9E−4 | 0.002 | 0.001 |
| 6-61.5 | 148039 | 151 | 494 | 0.120 | 7.6E−4 | 0.565 | 0.006 |
| 6-61.5 | 155861 | 160 | 503 | 0.082 | 7.2E−4 | 0.490 | 0.003 |
| 6-63.1 | 20410 | 32 | 377 | 0.028 | 0.012 | 0.055 | 0.138 |
| 6-66.6 | 8838 | 12 | 357 | 0.050 | 0.009 | 0.031 | 0.025 |
| 6-67.5 | 14694 | 24 | 369 | 0.226 | 8.2E−4 | 0.496 | 0.151 |
| 6-86.9 | 110972 | 130 | 475 | 0.023 | 0.050 | 0.072 | 0.482 |
| 6-110.4 | 31684 | 50 | 395 | 0.012 | 9.6E−4 | 0.162 | 0.240 |
| 6-121 | 37634 | 68 | 413 | 0.002 | 0.052 | 0.052 | 0.008 |
| 6-132.7 | 37555 | 67 | 412 | 0.089 | 0.364 | 0.665 | 0.025 |
| 7-62 | 42164 | 78 | 423 | 0.075 | 0.424 | 0.045 | 0.235 |
| 7-67 | 30674 | 46 | 391 | 0.424 | 0.048 | 0.187 | 0.015 |
| 7-68.7 | 39064 | 71 | 416 | 0.321 | 0.558 | 0.028 | 0.357 |
| 7-72.8 | 42930 | 177 | — | 0.111 | 0.076 | 0.006 | 0.002 |
| 7-74.2 | 68426 | 92 | 437 | 0.013 | 0.662 | 0.047 | 0.088 |
| 7-98.5 | 8799 | 11 | 356 | 0.031 | 0.429 | 0.009 | 0.160 |
| 7-98.8 | 48425 | 82 | 427 | 7.4E−4 | 0.063 | 2.6E−4 | 0.034 |
| 7-99.8 | 4415 | 163 | — | 6.9E−4 | 0.057 | 1.5E−4 | 0.032 |
| 7-99.8 | 35408 | 59 | 404 | 0.003 | 0.055 | 0.002 | 0.069 |
| 7-107.5 | 38914 | 70 | 415 | 0.024 | 0.002 | 0.682 | 0.747 |
| 7-115.8 | 4093 | 162 | — | 0.185 | 0.007 | 0.512 | 0.050 |
| 7-118.6 | 4302 | 6 | 351 | 0.032 | 6.5E−4 | 0.522 | 0.120 |
| 7-118.6 | 38653 | 175 | — | 0.199 | 0.011 | 0.471 | 0.035 |
| 7-118.6 | 81460 | 98 | 443 | 0.061 | 0.002 | 0.578 | 0.257 |
| 7-122.2 | 145260 | 143 | 488 | 0.062 | 0.003 | 0.108 | 0.003 |
| 7-124.5 | 15184 | 23 | 368 | 0.044 | 0.009 | 0.079 | 0.008 |
| 7-124.5 | 39773 | 74 | 419 | 0.065 | 0.022 | 0.814 | 0.608 |
| 7-132.8 | 30029 | 44 | 389 | 0.330 | 0.046 | 0.577 | 0.552 |
| 8-16.4 | 40320 | 76 | 421 | 0.657 | 0.063 | 0.405 | 0.006 |
| 8-40.9 | 107937 | 116 | 461 | 0.048 | 0.046 | 0.221 | 0.077 |
| 8-43.1 | 111628 | 132 | 477 | 0.105 | 0.011 | 0.401 | 0.144 |
| 8-45.5 | 26720 | 37 | 382 | 0.109 | 0.043 | 0.459 | 0.282 |
| 8-47.9 | 104862 | 106 | 451 | 0.152 | 0.143 | 0.011 | 0.276 |
| 8-53.9 | 27361 | 38 | 383 | 0.798 | 0.947 | 0.378 | 0.048 |
| 8-53.9 | 145200 | 142 | 487 | 0.260 | 0.129 | 0.033 | 0.016 |
| 8-55.7 | 23091 | 171 | — | 0.040 | 0.183 | 0.069 | 0.872 |
| 8-59.3 | 77568 | 179 | — | 0.003 | 0.258 | 4.4E−5 | 0.249 |
| 8-64 | 110148 | 127 | 472 | 0.005 | 0.085 | 0.005 | 0.528 |
| 8-65.8 | 104389 | 103 | 448 | 0.001 | 0.117 | 0.004 | 0.415 |
| 8-66.6 | 21895 | 170 | — | 0.003 | 0.143 | 0.002 | 0.417 |
| 8-67.4 | 48562 | 83 | 428 | 0.006 | 0.081 | 0.005 | 0.416 |
| 8-68.4 | 82295 | 100 | 445 | 0.003 | 0.311 | 4.9E−4 | 0.067 |
| 8-85.9 | 110684 | 128 | 473 | 0.039 | 0.588 | 0.030 | 0.082 |
| 8-87.5 | 9759 | 17 | 362 | 0.473 | 0.574 | 0.010 | 0.353 |
| 8-105.5 | 107286 | 115 | 460 | 0.028 | 0.063 | 0.104 | 0.076 |
| 8-106.8 | 13100 | 21 | 366 | 0.030 | 0.068 | 0.107 | 0.043 |
| 8-117.3 | 145077 | 141 | 486 | 0.006 | 0.009 | 0.055 | 0.192 |
| 8-117.3 | 145298 | 144 | 486 | 0.005 | 0.004 | 0.076 | 0.210 |
| 9-20.5 | 58904 | 178 | — | 0.578 | 0.351 | 0.078 | 0.041 |
| 9-80 | 29745 | 40 | 385 | 0.182 | 0.025 | 0.538 | 0.116 |
| 9-93.8 | 110377 | 183 | — | 0.021 | 0.097 | 0.019 | 0.134 |
| 9-93.8 | 113113 | 134 | 479 | 0.022 | 0.098 | 0.026 | 0.290 |
| 9-94 | 25961 | 173 | — | 0.058 | 0.047 | 0.039 | 0.177 |
| 9-94.5 | 148621 | 152 | 495 | 0.060 | 0.300 | 0.030 | 0.066 |
| 9-100.6 | 20048 | 29 | 374 | 0.034 | 0.137 | 0.155 | 0.156 |
| 9-100.6 | 153427 | 157 | 500 | 0.022 | 0.154 | 0.092 | 0.101 |
| 9-110.3 | 8937 | 13 | 358 | 0.014 | 0.652 | 0.035 | 0.233 |
| 9-125.2 | 9555 | 15 | 360 | 0.419 | 0.270 | 0.307 | 0.049 |
| 9-137.2 | 36022 | 60 | 405 | 0.136 | 0.014 | 0.290 | 0.096 |
| 10-52.7 | 143754 | 139 | 484 | 0.608 | 0.127 | 0.648 | 0.044 |
| 10-56.1 | 39275 | 72 | 417 | 0.061 | 0.019 | 0.054 | 0.085 |
| 10-89.6 | 106742 | 111 | 456 | 0.064 | 0.105 | 0.035 | 0.088 |
| 10-93.2 | 143657 | 138 | 483 | 0.560 | 0.046 | 0.790 | 0.186 |
| 10-93.2 | 145800 | 145 | 489 | 0.551 | 0.030 | 0.793 | 0.190 |
| 10-100.9 | 109666 | 125 | 470 | 0.083 | 0.007 | 0.144 | 0.148 |
| unmapped | 152577 | 158 | 501 | 0.315 | 0.001 | 0.627 | 0.914 |
| unmapped | 20742 | 30 | 375 | 0.425 | 0.141 | 0.435 | 0.041 |

### EXAMPLE 2

This example illustrates the preparation of a transgenic plant with a DNA construct for altered expression of an oil-associated gene.

Coding sequences of oil-associated genes are amplified by PCR prior to insertion in a GATEWAY™ Destination plant expression vector, as described in the detailed description. Primers for PCR amplification are designed at or near the start and stop codons of the coding sequence, in order to eliminate most of the 5′ and 3′ untranslated regions. PCR products are tailed with attB 1 and attB2 sequences in order to allow cloning by recombination into GATEWAY™ vectors (Invitrogen Life Technologies, Carlsbad, Calif.).

Corn callus is transformed by Agrobacterium-mediated methods well known in the art and regenerated to produce transgenic plants. Corn plants are grown in the greenhouse to maturity and reciprocal pollinations are made. Seed is collected from plants and used for further breeding activities. Tissue from the transgenic plant is assayed to verify the presence of the DNA construct and oil level in seed is measured to verify enhanced oil level.

### EXAMPLE 3

Homologs

A BLAST searchable “All Protein Database” was constructed of known protein sequences of plants and bacteria using a proprietary sequence database and the National Center for Biotechnology Information (NCBI) non-redundant amino acid database (nr.aa), which was filtered to contain only plants and bacteria based on NCBI division classification of sequences. A “Maize Protein Database” was constructed of known protein sequences of maize; it is a subset of the All Protein Database based on the NCBI taxonomy ID for maize.

The All Protein Database was queried using genomic DNA sequences of the oil-associated genes using “blastx” with E-value cutoff of 1e-8. Up to 200 top hits were kept and separated by organism names. For each organism other than maize, a list was kept for the hits from maize itself with a more significant E-value than the best hit of the organism. The list contains likely duplicated genes of the oil-associated gene and is referred as the Core List. Another list was kept for all the hits from each organism, sorted by the E-value, and is referred to as the Hit List

The Maize Protein Database was queried using DNA sequence of the oil-associated genes using “blastx” with E-value cutoff of 1e-4. Up to 200 top hits were kept. A BLAST searchable database was constructed based on these hits and is referred to as “SubDB,” which was queried with each sequence in the Hit List using “blastp” with E-value cutoff of 1e-8. The hit with best E-value was compared with the Core List of the corresponding organism. The hit was deemed a likely ortholog if it belonged to the Core List, otherwise it was deemed not a likely ortholog and there was no further search of sequences in the Hit List for the same organism. The above process was applied using the DNA sequences of the 186 amplicon of maize oil markers. 1955 likely orthologs from 357 distinct organisms were identified and reported by amino acid sequences of SEQ ID NO:505 to SEQ ID NO:2459. These 1955 likely orthologs are reported in Table 1 as homologs to 120 of the oil-associated genes.

All of the compositions and methods disclosed and claimed herein can be made and executed without undue experimentation in light of the present disclosure. Although the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

All publications and patent applications cited herein are incorporated by reference in their entirely to the same extent as if each individual publication or patent application was specifically and individually indicated to be incorporated by reference.

What is claimed is:

1. Transgenic plant seed having in its genome a recombinant DNA construct comprising at least one oil-associated gene operably linked to a promoter that is functional in said plant to transcribe said oil-associated gene.

2. Transgenic plant seed of claim 1 that grows into a plant having enhanced seed oil as compared to wild type.

3. Transgenic plant seed having in its genome a recombinant DNA construct comprising a gene suppression DNA operably linked to a promoter that is functional in said plant wherein transcription of said gene suppression DNA suppresses expression of an oil-associated gene.

4. Transgenic plant seed of claim 3 wherein transcription of said gene suppression DNA produces a dsRNA.

5. Hybrid maize seed that is produced by crossing two parental maize lines where at least one of said parental maize lines is a transgenic maize line that has in its genome a recombinant DNA construct comprising at least one oil-associated gene operably linked to a promoter that is functional in said plant to transcribe said oil-associated gene.

6. Hybrid maize seed of claim 5 wherein said parental maize lines are selected to produce maize plants characterized by agronomic traits where seed oil level for said maize plants is greater than seed oil level in the parental lines.

7. Hybrid maize seed of claim 5 wherein said parental maize lines are selected to produce maize plants characterized by agronomic traits where there is essentially no reduction in yield and standability traits in said maize plants as compared to the parental lines.

8. Hybrid maize seed according to claim 5 having in its genome recombinant DNA constructs for expressing 2 or more oil-associated genes.

9. A method of producing hybrid maize plants having enhanced levels of seed oil as compared to the closest non-transgenic ancestor maize hybrid for said plants, wherein the method comprises producing a transgenic maize plant having in its genome a recombinant DNA construct comprising at least one oil-associated gene operably linked to a promoter that is functional in maize to transcribe said oil-associated gene and crossing transgenic progeny of said transgenic maize plant with at least one other maize plant to produce said hybrid maize plants having enhanced levels of seed oil.

10 A method of breeding maize with higher oil comprising selecting from a breeding population of maize plants one or more selected maize plants based on allelic polymorphisms at one or more maize oil markers associated by linkage disequilibrium to genes conferring a higher seed oil-related trait, wherein the selected maize plant has 1 or more higher oil alleles linked to a maize oil marker.

11. A method of breeding maize according to claim 10 wherein said selected maize plant has 2 or more higher oil alleles linked to a maize oil marker.

12. A method of breeding maize according to claim 10 wherein said selected maize plant has 3 or more higher oil alleles linked to a maize oil marker.

13. A method of breeding maize comprising selecting a maize line having a haplotype characterized by one or more of the maize oil markers.

14. A recombinant DNA construct comprising a promoter functional in plants operably linked to an oil-associated gene.

15. A polymorphic maize DNA locus that is useful for genotyping between at least two varieties of maize; wherein said locus comprises at least 20 consecutive nucleotides that include or are adjacent to a maize oil marker; and wherein the sequence of said at least 20 consecutive nucleotides is at least 90% identical to the sequence of the same number of nucleotides in either strand of a segment of maize DNA that includes or is adjacent to said marker.

16. An isolated nucleic acid molecule useful for detecting a polymorphism associated with oil in maize, wherein said nucleic acid molecule comprises at least 12 nucleotide bases and a detectable label, and wherein the sequence of said at least 12 nucleotide bases is at least 90 percent identical to a sequence of the same number of consecutive nucleotides in either strand of a segment of maize DNA in a locus of claim 15.

17. A pair of nucleic acid molecules wherein each nucleic acid molecule of said pair is a nucleic acid molecule according to claim 16, and wherein each of said molecules has a distinct fluorescent dye at the 5′ end thereof and has identical nucleotide sequence except for a single nucleotide polymorphism.

18. An isolated nucleic acid molecule useful for detecting a polymorphism in maize DNA, wherein said nucleic acid molecule comprises at least 15 nucleotide bases, wherein the sequence of said at least 15 nucleotide bases is at least 90 percent identical to a sequence of the same number of consecutive nucleotides in either strand of a segment of maize DNA in a locus of claim 15.

19. A method of breeding maize comprising selecting a maize plant having a polymorphism associated by linkage disequilibrium to a seed oil-related trait wherein said polymorphism is linked to a locus of claim 15.

20. A method of associating a seed oil-related trait to a genotype in maize comprising
(a) identifying a set of one or more seed oil related traits characterizing said maize plants,
(b) selecting tissue from at least two maize plants having allelic DNA and assaying DNA or mRNA from said tissue to identify the presence or absence of a set of distinct polymorphisms comprising at least one polymorphism linked to a locus of claim 15, and
(c) identifying associations between said set of polymorphisms and said set of traits.
